(19) United States
(12) Patent Application Publication
(10) Pub. No.: US 2013/0029904 A1
(43) Pub. Date: Jan. 31, 2013

(54) HCV COMBINATION THERAPY

(71) Applicant: George Kukolj, Mont-Royal (CA)

(72) Inventor: George Kukolj, Mont-Royal (CA)

(73) Assignee: BOEHRINGER INGELHEIM INTERNATIONAL GMBH, Ingelheim am Rhein (DE)

(21) Appl. No.: 13/515,967

(22) PCT Filed: Dec. 13, 2010
(86) PCT No.: PCT/CA2010/001935

§ 371 Date: Aug. 6, 2012

Related U.S. Application Data
(60) Provisional application No. 61/288,004, filed on Dec. 18, 2009.
(60) Provisional application No. 61/317,343, filed on Mar. 25, 2010.

(51) Int. Cl. A61K 38/05; A61P 31/14

(52) U.S. Cl. 514/4.3

(57) ABSTRACT

Aspects of this invention include methods comprising administering a combination of a Compound (1) below, including particular en crystalline forms thereof and pharmaceutically acceptable salts thereof, with at least one further selected HCV inhibiting compound as described herein for the treatment of Hepatitis C Viral (HCV) infection The methods can be conducted administering the Compound (1) and the at least one further selected HCV inhibiting compound separately or together, including as a regimen of treatment.

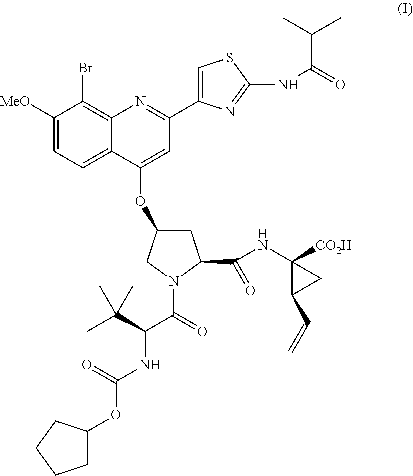 [SMILES: C=C[C@@H]1C[C@]1(NC(=O)[C@@H]1C[C@H](Oc2cc(-c3csc(NC(=O)C(C)C)n3)nc3c(Br)c(OC)ccc23)CN1C(=O)[C@@H](NC(=O)OC1CCCC1)C(C)(C)C)C(=O)O]

### RELATED APPLICATIONS

This application claims benefit of U.S. Ser. No. 61/288,004 filed Dec. 18, 2009, and U.S. Ser. No. 61/317,343 filed Mar. 25, 2010 both of which are herein incorporated by reference.

### FIELD OF THE INVENTION

The present invention relates to therapeutic combinations comprising Compound (1) as herein described, or a pharmaceutically acceptable salt thereof, with at least one further selected HCV inhibiting compound as described below for the treatment of Hepatitis C Viral (HCV) infection. The present invention also relates to methods of using such therapeutic combinations for treating HCV infection or alleviating one or more symptoms thereof in a patient, where each compounds is administered either together or separately. The present invention also provides kits comprising the therapeutic combinations of the present invention.

### BACKGROUND OF THE INVENTION

Hepatitis C virus (HCV) infection is a global human health problem with approximately 150,000 new reported cases each year in the United States alone. HCV is a single stranded RNA virus, which is the etiological agent identified in most cases of non-A, non-B post-transfusion and post-transplant hepatitis and is a common cause of acute sporadic hepatitis. It is estimated that more than 50% of patients infected with HCV become chronically infected and 20% of those develop cirrhosis of the liver within 20 years.

Several types of interferons, in particular, alfa-interferons are approved for the treatment of chronic HCV, e.g., interferon-alfa-2a (ROFERON®-A), interferon-alfa-2b (INTRON®-A), consensus interferon (INFERGEN®), as well as pegylated forms of these and other interferons like pegylated interferon alfa-2a (PEGASYS®) and pegylated interferon alfa-2b (PEG-INTRON®). Ribavirin, a guanosine analog with broad spectrum activity against many RNA and DNA viruses, has been shown in clinical trials to be effective against chronic HCV infection when used in combination with interferon-alfas (see, e.g., Poynard et al., Lancet 352:1426-1432, 1998; Reichard et al., Lancet 351:83-87, 1998), and this combination therapy has been approved for the treatment of HCV: REBETRON® (interferon alfa-2b plus ribavirin, Schering-Plough); PEGASYS®RBV® (pegylated interferon alfa-2a plus ribavirin combination therapy, Roche); see also Manns et al, Lancet 358:958-965 (2001) and Fried et al., 2002, N Engl. J. Med. 347:975-982. However, even with this combination therapy the sustained virologic response rate among patients chronically infected with genotype I is still at or below 50%.

The interferons require administration by injection, which is a much less preferred mode of administration from the standpoint of patient compliance and convenience. Furthermore, there are significant side-effects typically associated with such therapies. Ribavirin suffers from disadvantages that include teratogenic activity, interference with sperm development, haemolysis, anemia, fatigue, headache, insomnia, nausea and/or anorexia. Interferon alfa, with or without ribavirin, is associated with many side effects. During treatment, patients must be monitored carefully for flu-like symptoms, depression, rashes and abnormal blood cell counts. Patients treated with interferon alfa-2b plus ribavirin should not have complications of serious liver dysfunction and such subjects are only considered for treatment of hepatitis C in carefully monitored settings.

Effective and durable therapies that adequately suppress HCV replication typically require combinations of agents that target different biological mechanisms related to HCV infection. There is continuing need in the field for an effective combination therapy against HCV that avoids the inconvenience and significant side-effects typically associated with the available interferon plus ribavirin combination therapies.

The following Compound (1):

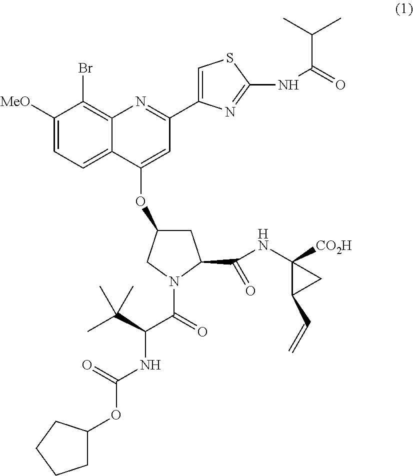 [SMILES: C=C[C@@H]1C[C@]1(NC(=O)[C@@H]1C[C@H](Oc2cc(-c3csc(NC(=O)C(C)C)n3)nc3c(Br)c(OC)ccc23)CN1C(=O)[C@@H](NC(=O)OC1CCCC1)C(C)(C)C)C(=O)O]

is known as a selective and potent inhibitor of the HCV NS3 serine protease for the treatment of HCV infection. Compound (1) falls within the scope of the acyclic peptide series of HCV inhibitors disclosed in U.S. Pat. Nos. 6,323,180, 7,514,557 and 7,585,845. Compound (1) is disclosed specifically as Compound #1055 in U.S. Pat. No. 7,585,845, and as Compound #1008 in U.S. Pat. No. 7,514,557. Compound (1) can be prepared according to the general procedures found in the above-cited references, which are herein incorporated by reference. Preferred forms of Compound (1) include the crystalline forms, in particular the crystalline sodium salt form, which can be prepared as described in the examples section herein.

Compound (1) may also be known by the following alternate depiction of its chemical structure, which is equivalent to the above-described structure:

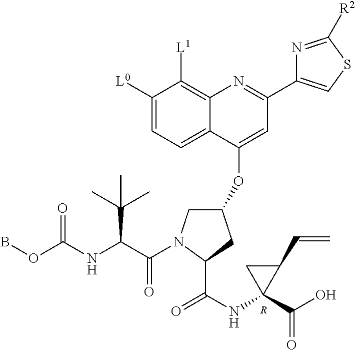

wherein B is

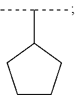

### L⁰ is MeO—; L¹ is Br; and R² is

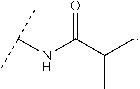

The following compounds (A) to (U) are also known compounds that are useful for the treatment of HCV infection (for clarification, any open valence on any nitrogen or oxygen atom in the structures depicted below should be considered filled by hydrogen):

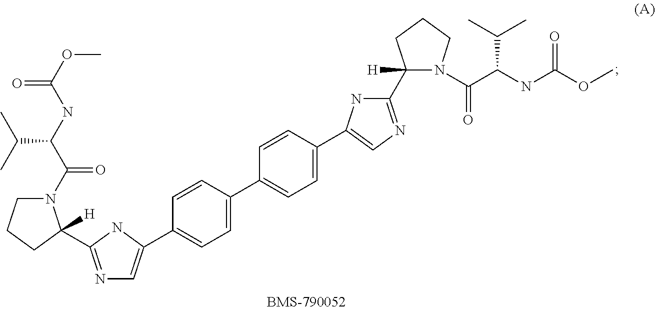 [SMILES: COC(=O)N[C@H](C(=O)N1CCC[C@H]1c1ncc(-c2ccc(-c3ccc(-c4cnc([C@@H]5CCCN5C(=O)[C@@H](NC(=O)OC)C(C)C)[nH]4)cc3)cc2)[nH]1)C(C)C]

BMS-790052 (Bristol Myers Squibb) is described, for example, in WO Pub. No. 2008/021927, WO Pub. No. 2008/021928 and WO Pub. No. 2009/020828.

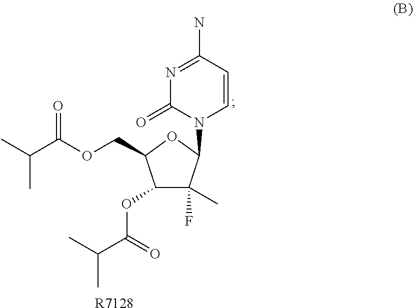

Chemical Name: 2′-Deoxy-2′(R)-fluoro-2′-methyl-3′,5′-di-O-isobutyrylcytidine. R7128 or RG7127 (Roche) is a prodrug of PSI-6130 shown below:

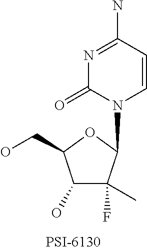 [SMILES: C[C@@]1(F)[C@H](O)[C@@H](CO)O[C@H]1n1ccc(N)nc1=O]

R7128, PSI-6130 and related compounds, their properties and synthesis are described, for example, in WO Pub. No. WO2005003147, and also in Drugs of the Future, 2009, 34 (4): pg 282-290; Drugs, 2009, 69, 2, pg 151-166 (see FIG. 6 pg 159), Clark, J. L. et al., J. Med. Chem., 2005, 48, 5504; and Stuyver, L. J. et al., Antiviral Chemistry and Chemotherapy, 2006, 17, 79.

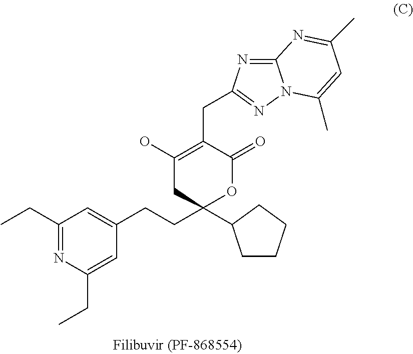 [SMILES: CCc1cc(CC[C@]2(C3CCCC3)CC(O)=C(Cc3nc4nc(C)cc(C)n4n3)C(=O)O2)cc(CC)n1]

Chemical Name: 6(R)-Cyclopenyl-6-[2-(2,6-diethylpyridin-4-yl)ethyl]-3-(5,7-dimethyl[1,2,4]triazolo[1,5-a]pyrimidin-2-ylmethyl)-4-hydroxy-5,6-dihydro-2H-pyran-2-one (CAS Registry No. 877130-28-4). Filibuvir or PF-868554 (Pfizer) is described, for example, in WO Pub. No. 2006/018725, and also in J Med Chem. 2009, 52, 5, pg 1255-1258 (see the top of pg 1257, Table 3, Compound 24)), Annual Report in Med Chem, Vol 44, 2009, pg 397-440 (see pg. 416, Compound 40), and Antimicrobrial Agents and Chemotherapy, 2009, 53, 6, pg 2544-2552 (see the top of pg. 2545).

(D) The HCV non-nucleoside polymerase inhibitor compound known by the trade name ANA-598 (Anadys Pharmaceuticals Inc.). ANA-598 and related structures and syntheses are described in WO Pub. Nos. 2006/066079 and 2006/066080, 2007/150001, 2008/124450 and 2010/042834 See also Hepatology, 48(4, Suppl. S), October 2008, 1026A, 1163A and 1164A.

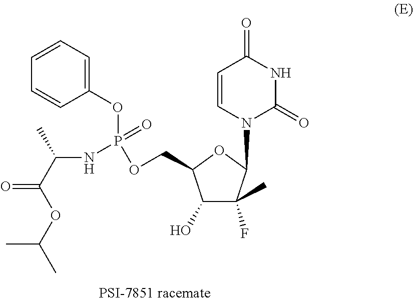 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

PSI-7851 (Pharmasset) is described, for example, in A M Lam et al. Global Antiviral Journal, Vol 5, Supplement 1, page 137-138, 2009 (Abstracts 103 and 152); Annual Reports in Medicinal Chemistry, Volume 44, 2009, Chapter 20, Pages 397-440; and in Furman, P. A., et. al., 15^{th} International Symposium on HCV & Related Viruses, San Antonio, Tex., Oct. 5-9, 2008 (Abstract #275). PSI-7851 is a racemic mixture of two isomers PSI-7976 and PSI-7977 PSI-7851 is a prodrug of PSI-7409 shown below:

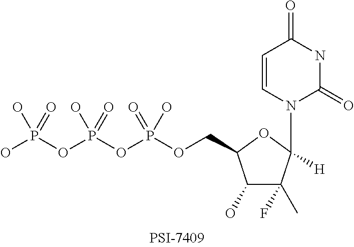 [SMILES: C[C@@]1(F)[C@H](O)[C@@H](COP(=O)(O)OP(=O)(O)OP(=O)(O)O)O[C@H]1n1ccc(=O)[nH]c1=O]

PSI-7977 is also a prodrug of the nucleotide analog PSI-7409. Related compounds and syntheses are described, for example, in WO Pub. No. 2005/003147. PSI-7977 is the single diastereomer as described by Sofia M J, Bao D, Chang W, Du J, Nagarathnam D, Rachakonda S, Reddy P G, Ross B S, Wang P, Zhang H R, Bansal S, Espiritu C, Keilman M, Lam A M, Steuer H M, Niu C, Otto M J, Furman P A in J Med Chem. 2010 Oct. 14; 53(19):7202-18.

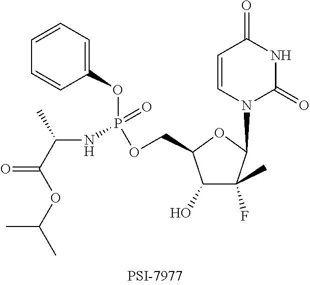

(F) The HCV polymerase inhibitor compound known by the trade name IDX 184 (Idenix Pharmaceuticals Inc.) is a prodrug of 2′-methyl guanosine. IDX 184 and its properties, related compounds and syntheses are described, for example, in Cretton-Scott, E. et al., European Association for the Study of the Liver, 43^{rd} Annual Meeting, Milan Italy, Apr. 23-27 2008 (Abstract #588), J. Hepatology: 50(Suppl. 1). 2009. S37; 48(Suppl. 2). 2008. S220: 48(Suppl. 2). 2008. S30, WO Pub. Nos. 2008/082601 and 2010/014134.

(G) VX-222 or VCH-222 (Vertex Pharmaceuticals Inc.) is a non-nucleoside HCV polymerase inhibitor compound known by the trade name. VX-222 and related compounds and syntheses are described, for example, in EP Pub. No. 1321463 and Cooper C. et al., J. Hepatology: 50(Suppl. 1), 2009, S340: 50, Abs939, Suppl. 1, 2009 and 50, Abs940, Suppl. 1, 2009.

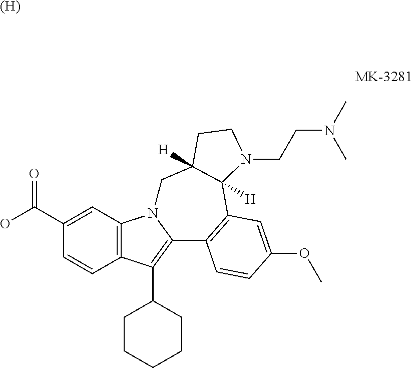 [SMILES: "(H)" ; COc1ccc2c(c1)[C@H]1[C@@H](CCN1CCN(C)C)Cn1c-2c(C2CCCCC2)c2ccc(C(=O)O)cc21]

MK-3281 (Merck & Co.) is described, for example, in WO Pub. No. 2007/129119.

(I) AZD7295 or A-689 (AstraZeneca plc) is described, for example, in Expert Opinion on Drug Discovery, 4(3)(pp 293-314), March 2009: Recent Patents on Anti-Infective Drug Discovery, 3(2) (pp 77-92), 2008: and Clinics in Liver Disease, 12(3), pp. 529-555 (2008).

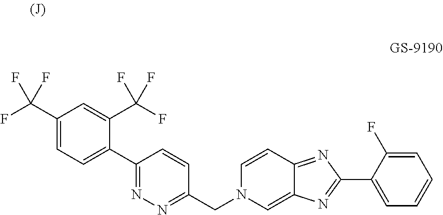 [SMILES: Fc1ccccc1-c1nc2ccn(Cc3ccc(-c4ccc(C(F)(F)F)cc4C(F)(F)F)nn3)cc-2n1]

GS-9190 (Gilead) and related compounds and syntheses are described, for example, in WO Pub. Nos. 2005/063744, 2008/005519 and 2009/009001; Annual Reports in Medicinal Chemistry, Volume 44, 2009, Chapter 20, Pages 397-440 (see pg. 419-420, Structure 48); and Yang, C. et al., American Association for the Study of Liver Diseases, 58^{th} Annual Meeting, Boston, Nov. 2-6, 2007 (Abstract #1398).

(K) ABT-333 (Abbott Laboratories) is a non-nucleoside HCV polymerase inhibitor compound described, for example, in Koev, G. et al., J. Hepatology, 50(Suppl. 1). 2009. S346-S348; Expert Opinion on Therapeutic Patents, 19(2) (pp 145-164), February 2009; and Clinics in Liver Disease, 13(3) (pp 453-465), August 2009.

(L) ABT-072 (Abbott) is a non-nucleoside HCV polymerase inhibitor compound described, for example, in Koev, G. et al., J. Hepatology, 50(Suppl. 1), 2009, S346-S348 and S352.

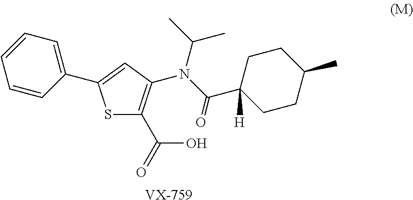

VX-759 (Vertex) is a non-nucleoside HCV polymerase inhibitor compound described, for example, in Bioorg. & Med. Chem. Letters, 14 (2004), 797-800; Cooper C. et al., J. Hepatology 51 (2009) 39-46 where it is referred to by its former reference VCH-759 and in WO Pub. No. 2002/100851. Similar compounds are described in WO Pub. No. 2004/052885.

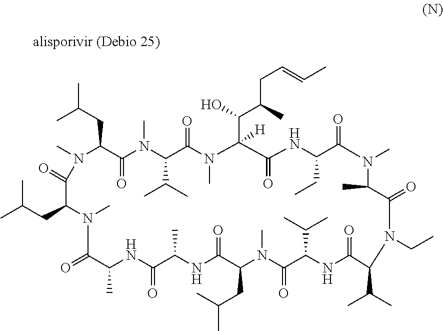 [SMILES: C/C=C/C[C@@H](C)[C@@H](O)[C@H]1C(=O)N[C@@H](CC)C(=O)N(C)[C@H](C)C(=O)N(CC)[C@@H](C(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

Alisporivir (Debiopharm) or Debio 25 is a cyclophilin inhibitor described, for example, in WO Pub. No. 2000/001715, WO Pub. No. 2006/038088; Coelmont et al. Antimicrobial Agents and Chemotherapy, Vol 53, No. 3, 967-976 (March 2009); and Herrmann, E. et al., J. Hepatology, 2009, 50, S344.

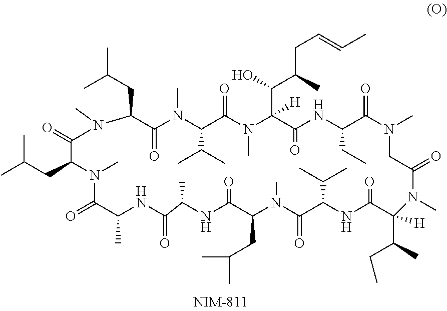 [SMILES: C/C=C/C[C@@H](C)[C@@H](O)[C@H]1C(=O)N[C@@H](CC)C(=O)N(C)CC(=O)N(C)[C@@H]([C@@H](C)CC)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

NIM-811 (Novartis) is a cyclophilin inhibitor. NIM-811 and related compounds and syntheses are described, for example, in WO Pub. No. 2006/071619; WO Pub. No. 2006/071618; Mathy et al., Antimicrobial Agents and Chemotherapy, Vol 52, No. 9, 3267-3275 (September 2008); Lawitz, E. et al., J. Hepatology, 2009, 50, S379.

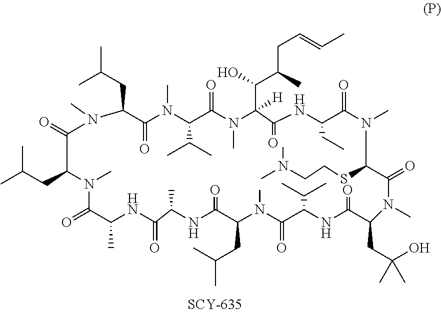 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

SCY-635 (Scynexis) is a cyclophilin inhibitor. SCY-635 and related compounds and syntheses are described, for example, in WO Pub. No. 2006/039668; WO Pub. No. 2009/828330; and Chatterji U. et al., J. Biol. chem., 2009, 284, 16998.

(Q) BMS-791325 is an HCV replication inhibitor in clinical trials for the treatment of HCV infected patients, as reported in the US National Institutes of Health clinical trials database (http://clinicaltrials.gov/ct2/show/NCT00664625).

(R) BMS-824393 is a HCV NS5A inhibitor in clinical trials for the treatment of HCV infected patients, as reported in the US National Institutes of Health clinical trials database (http://clinicaltrialsfeeds.org/clinical-trials/show/NCT00971308). BMS-824393 is described in Abstract 1858 (Nettles R. E et al.) presented at the 61^{st} Annual Meeting of the American Association for the Study of Liver Diseases (AASLD) meeting in Nov. 2, 2010 (Boston).

(S) PSI-938 (Pharmasset), also known as PSI-352938, is a β-D-2′-deoxy-2′-fluoro-2′-C-methylpurine monophosphate prodrug in clinical trials for the treatment HCV infected patients. PSI-938 is described in Abstract 1890 (Symonds et al.) presented at the 61^{st} Annual Meeting of the American Association for the Study of Liver Diseases (AASLD) meeting in Nov. 2, 2010 (Boston).

(T) PPI-461 (Presidio) is an HCV replication inhibitor in clinical trials for the treatment of HCV infected patients, as reported in US National Institutes of Health clinical trials database; at the 46^{th} annual meeting of the European Association for the Study of the Liver (EASL), Mar. 30-Apr. 3, 2011, Berlin, Germany; and the 61th annual meeting of the American Association for the Study of Liver Diseases, Oct. 30-Nov. 3, 2010.

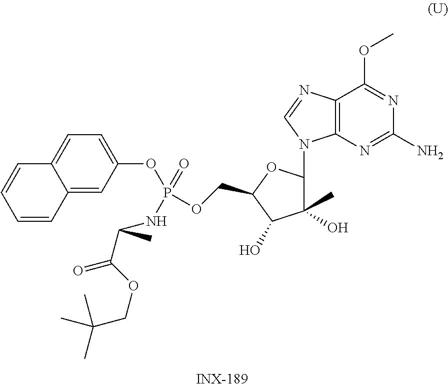 [SMILES: COc1nc(N)nc2c1ncn2C1O[C@H](COP(=O)(N[C@H](C)C(=O)OCC(C)(C)C)Oc2ccc3ccccc3c2)[C@@H](O)[C@@]1(C)O]

NX-189 (Inhibitex) is a novel potent phosphoramidate based pro-drug of an 06-methyl modified 2′-C Methyl guanosine monophosphate currently in clinical development for the treatment of HCV. INX-189 and its properties are described, for example, in 61st Annual Meeting of the American Association for the Study of Liver Diseases (Abstract #1874) and the US National Institutes of Health clinical trials database http://www.clinicaltrials.gov/ct2/show/NCT01159808.

### SUMMARY OF THE INVENTION

One embodiment of the invention is directed to a method of treating HCV infection in a mammal comprising administering to said mammal a therapeutically effective amount of each of:
(a) Compound (1) as described above, including crystalline forms thereof, or a pharmaceutically acceptable salt thereof; and
(b) one or more of the following further HCV inhibiting compounds (A)-(U), as described above:
   (A) BMS-790052
   (B) R7128
   (C) filibuvir (PF-868554)
   (D) ANA-598
   (E) PSI-7851 racemate, or PSI-7977 or PSI 7976 isomers
   (F) IDX 184
   (G) VX-222
   (H) MK-3281
   (I) AZD 7295
   (J) GS-9190
   (K) ABT-333
   (L) ABT-072
   (M) VX-759
   (N) alisporivir (Debio 25)
   (O) NIM-811
   (P) SCY-635
   (Q) BMS-791325
   (R) BMS-824393
   (S) PSI-938
   (T) PPI-461
   (U) INX-189
or a pharmaceutically acceptable salt thereof.

Individual and separate embodiments of the invention include the sixteen different combinations obtained by combining Compound (1), or a pharmaceutically acceptable salt thereof, with each of the individual compounds (A) to (P), or pharmaceutically acceptable salt thereof. Further embodiments of the invention include the combinations obtained by combining Compound (1), or a pharmaceutically acceptable salt thereof, with each of the individual compounds (Q) to (U) or pharmaceutically acceptable salt thereof.

In another embodiment the invention is directed to a method of treating HCV infection in a mammal comprising administering to said mammal a therapeutically effective amount of each of:

(a) Compound (1) as described above, including crystalline forms thereof, or a pharmaceutically acceptable salt thereof; and

(b) one or more of the following further HCV inhibiting compounds selected from the group consisting of BMS-790052, R7128, filibuvir (PF-868554), ANA-598, PSI-7851, PSI-7977, PSI-7976, IDX 184, VX-222, MK-3281, AZD 7295, GS-9190, ABT-333, ABT-072, VX-759, alisporivir (Debio 25), NIM-811, SCY-635, PSI-7977, BMS-791325, BMS-824393, PSI-938, PPI-461 and INX-189 or a pharmaceutically acceptable salt thereof.

The Compound (1) can be used in any crystalline form thereof, and the pharmaceutically acceptable salts of Compound (1) may also be in crystalline form. A particularly preferred salt of Compound (1) is the sodium salt form. The recitation “Compound (1)” or “pharmaceutically acceptable salt of Compound (1)” as used herein thus embraces any crystalline form of these compounds.

The compounds (A)-(U) may also be used in any particular isomeric form (e.g., enantiomers, stereoisomers, diastereomers, tautomers, racemates, etc) where possible, or in a crystalline form or in a pharmaceutically acceptable salt form thereof. For example, compound (E) may be either the PSI-7851 racemate or one of its isomers PSI-7977 and PSI 7976. Thus, unless otherwise defined by the chemical structure provided, the recitation of (A) to (U), or pharmaceutically acceptable salt forms thereof, as used herein thus embraces any isomeric or crystalline form of these compounds. For further clarification, the chemical structures for compounds (A) to (U), where provided in the present application, are believed to be the correct structure for the compound. Nevertheless, in the event the actual structure for any of the named anti-HCV development candidates (A) to (U) differ from the structures provided herein, the actual chemical structure controls and is considered herein incorporated by reference.

Another embodiment is directed to the above-described method wherein the Compound (1) or pharmaceutically acceptable salt thereof, and two or more (e.g., two, three or four) of the further HCV inhibiting compounds (A)-(U), or a pharmaceutically acceptable salt thereof, or mixtures thereof, are administered. As appreciated by those skilled in the art a combination of Compound (1) with two or more of the further HCV inhibiting compounds (A)-(U) would preferably select two or more further HCV inhibiting compounds having distinct resistance profiles or whose mechanism of action involves distinct HCV binding pockets.

Another embodiment is directed to the above-described method wherein, in addition to administering the Compound (1), or pharmaceutically acceptable salt thereof, and one or more of the further HCV inhibiting compounds (A)-(U), or pharmaceutically acceptable salt thereof, or mixtures thereof, there is also administered one or more additional compounds for HCV inhibition. The additional compound for HCV inhibition can be, for example, an interferon or ribavirin or a combination thereof. As examples for the additional interferon, several types of interferons (eg. alfa-interferons, lambda interferons, omega interferon), in particular, alfa-interferons are approved for the treatment of chronic HCV, e.g., interferon-alfa-2a (ROFERON®-A), interferon-alfa-2b (INTRON®-A), consensus interferon (INFERGEN®), fusion products of human albumin and interferon alfa (Abuferon®), as well as pegylated forms of these and other interferons like pegylated interferon alfa-2a (PEGASYS®) pegylated interferon alfa-2b (PEG-INTRON®), and PEG-interferon lambda may be used. Ribavirin is a guanosine analog with broad spectrum activity against many RNA and DNA viruses and has been shown in clinical trials to be effective against chronic HCV infection when used in combination with interferon-alfas (see, e.g., Poynard et al., Lancet 352:1426-1432, 1998; Reichard et al., Lancet 351:83-87, 1998). Certain interferon-containing combination therapies for treating HCV infection are also disclosed in the following U.S. Patent Application Publications: US 2005/0112093; US 2005/0129659; and US 2008/0138316.

Another embodiment is directed to a package comprising one or more doses of a Compound (1) or a pharmaceutically acceptable salt thereof, and instructions directing the administration of Compound (1) or a pharmaceutically acceptable salt thereof and one or more of the further HCV inhibiting compounds (A)-(U), or a pharmaceutically acceptable salt thereof, for the treatment of HCV infection.

Another embodiment of the invention is directed to a pharmaceutical composition comprising a Compound (1), or pharmaceutically acceptable salt thereof, combined with one or more of the further HCV inhibiting compounds (A)-(U), or pharmaceutically acceptable salt thereof, or mixtures thereof, and at least one pharmaceutically acceptable carrier or diluent.

Another embodiment is directed to a pharmaceutical composition comprising a Compound (1), or a pharmaceutically acceptable salt thereof, combined with two or more (e.g., two, three or four) of the further HCV inhibiting compounds (A)-(U), or a pharmaceutically acceptable salt thereof, or mixtures thereof, and at least one pharmaceutically acceptable carrier or diluent.

Another embodiment of the invention is directed to a kit which separately comprises one or more doses of Compound (1), or a pharmaceutically acceptable salt thereof, and separately, one or more doses of one or more of the further HCV inhibiting compounds (A)-(U), or a pharmaceutically acceptable salt thereof, or mixtures thereof, packaged together. The kit may also include instructions for administering the doses to effect at least one of the above-described combination therapy methods.

### DETAILED DESCRIPTION OF THE INVENTION

### Definitions

Terms not specifically defined herein should be given the meanings that would be given to them by one of skill in the art in light of the disclosure and the context. As used throughout the present application, however, unless specified to the contrary, the following terms have the meaning indicated:

The term “pharmaceutically acceptable” with respect to a substance as used herein means that substance which is, within the scope of sound medical judgment, suitable for use in contact with the tissues of humans and lower animals without undue toxicity, irritation, allergic response, and the like, commensurate with a reasonable benefit/risk ratio, and effective for the intended use when the substance is used in a pharmaceutical composition.

The term “treating” with respect to the treatment of a disease-state in a patient include
(i) inhibiting or ameliorating the disease-state in a patient, e.g., arresting or slowing its development; or
(ii) relieving the disease-state in a patient, i.e., causing regression or cure of the disease-state. In the case of HCV, treatment includes reducing the level of HCV viral load in a patient.

Compound (1)

When synthesized according to the general procedures set forth in U.S. Pat. Nos. 6,323,180, 7,514,557 and 7,585,845, Compound (1) is prepared as an amorphous solid. But Compound (1) can also be produced in a crystalline form which may be preferable for particular pharmaceutical requirements and specifications. Furthermore, it is preferable that the process by which Compound (1) is produced is amenable to large-scale production. Additionally, it is preferable that the product should be in a form that is readily filterable and easily dried. Finally, it is economically preferable that the product be stable for extended periods of time without the need for specialized storage conditions.

In one embodiment, Compound (1) is used in crystalline form, salt form, or crystalline salt form. Thus, in one embodiment, the present invention provides Compound (1) for the methods and compositions in a crystalline form which is a crystalline polymorph designated herein as Type A, or also in the form of a crystalline salt of Compound (1). For the salt forms, sodium salts are preferred but other pharmaceutically acceptable salt forms can be used. The crystalline forms can be preferable for pharmaceutical processing issues.

The Type A crystalline form of Compound (1) exhibits a characteristic X-ray powder diffraction (XRPD) pattern with characteristic peaks expressed in degrees 2θ (±0.2 degrees 2θ) at 4.8, 6.8, 9.6, 13.6, 17.3, 19.8 and 24.5 measured using CuKα radiation.

The characteristic peak positions and relative intensities for the XRPD pattern of Type A is shown in Table 1 below.

TABLE 1
Compound (1)
Type A

| Angle | |
|---|---|
| 2-Theta ° | Rel. Intensity % |
| 4.8 | 100 |
| 6.8 | 6 |
| 9.6 | 24 |
| 13.6 | 6 |
| 17.3 | 8 |
| 19.8 | 16 |
| 24.5 | 11 |

The term “Type A” as used herein means a crystalline polymorph of Compound (1) that has an X-ray powder diffraction pattern having at least a characteristic peak at 9.6 degrees 2θ (±0.2 degrees 2θ) when measured using CuKα radiation. This characteristic peak is believed to distinguish Type A from other crystalline forms of Compound (1).

Thus, one specific embodiment is directed to the above-described methods and compositions wherein Compound (1) is in the form of a crystalline polymorph that has at least the following characteristic: an X-ray powder diffraction pattern comprising a peak at 9.6 degrees 2θ (±0.2 degrees 2θ) when measured using CuKα radiation.

Another embodiment is directed to the above-described methods and compositions wherein Compound (1) is in the form of a crystalline polymorph having an XRPD pattern comprising a peak at 9.6 degrees 2θ (±0.2 degrees 2θ) as described above and further comprising a peak at 19.8 degrees 2θ (±0.2 degrees 2θ) when measured using CuKα radiation.

Another embodiment is to the above-described methods and compositions wherein Compound (1) is in the form of a crystalline polymorph having an XRPD pattern comprising a peak at 9.6 degrees 2θ (±0.2 degrees 2θ) as described above and further comprising peaks at 4.8 and 19.8 degrees 2θ (±0.2 degrees 2θ) when measured using CuKα radiation.

Another embodiment is directed to the above-described methods and compositions wherein Compound (1) is in the form of a crystalline polymorph having an XRPD pattern comprising a peak at 9.6 degrees 2θ (±0.2 degrees 2θ) as described above and further comprising peaks at 4.8, 6.8, 13.6, 17.3, 19.8 and 24.5 degrees 2θ (±0.2 degrees 2θ) when measured using CuKα radiation.

Another embodiment is directed to the above-described methods compositions wherein at least 50%, preferably at least 75%, more preferably at least 95%, more preferably at least 99%, of said Compound (1) is present in crystalline form, for example, in the form of the Type A crystalline polymorph as characterized by any of the abovementioned XRPD-defined embodiments. The presence of such amounts of Type A polymorph in a quantity of Compound (1) is typically measurable using XRPD analysis of the compound.

The Type A polymorph can be prepared by a method which comprises crystallizing Compound (1) from a solution in solvents under conditions which yield Type A. The precise conditions under which Type A is formed may be empirically determined and the following methods are described which have been found to be suitable in practice.

For example, the Type A polymorph of Compound (1) may be prepared by a process comprising the following steps:
(i) dissolving Compound (1) in an aliphatic alcohol solvent, optionally containing water as a co-solvent, by heating the mixture to a temperature of about 65 to 75° C. to obtain a solution;
(ii) adding water to the solution obtained in step (i) while maintaining the solution at a temperature of about 70 to 75° C. to obtain a slurry;
(iii) cooling the slurry obtained in step (ii) to obtain solid material;
(iv) collecting the solid material of step (iii) and drying said material at a temperature of about 65 to 80° C. to obtain Type A of Compound (1).

Aliphatic alcohols that may be employed in this process include, for example, ethanol (e.g., denatured, 200 proof or 100% pure), 1-propanol, 2-propanol, 1-butanol, iso-butyl alcohol and iso-pentyl alcohol, preferably ethanol. The resulting crystals of Type A may be recovered by any conventional methods known in the art.

In the final step (iv), the resulting solids obtained in step (iii) may be collected and dried at high temperature using conventional collection and high-temperature drying techniques, for example, filtration and vacuum oven.

In one preferred embodiment of the preparation, amorphous Compound (1) is dissolved in an aliphatic alcohol solvent (e.g., ethanol), containing up to about 10% v/v water as co-solvent, by stirring and heating the mixture to a temperature of about 72 to 74° C. until Compound (1) completely dissolves. A separate water addition solution is prepared containing water and up to about 10% v/v aliphatic alcohol (e.g., ethanol), and this water addition solution is added approximately linearly over time to the Compound (1) solution while maintaining the mixture at a temperature of about 72 to 74° C. Type A of Compound (1) begins to crystallize during the addition of the water solution. The resulting crystal slurry is cooled and stirred, and the crystals are then filtered, washed and dried at a temperature of about 65 to 75° C. using conventional techniques.

The process steps may of course be facilitated by conventional agitation techniques, e.g., stirring, and other conventional techniques as would be well understood for facilitation the process.

As the Compound (1) component, the sodium salt of the Compound of formula (1) has been found to be preferable for pharmaceutical processing due to the fact that it can be prepared as a stable crystalline form. In general, the crystalline sodium salt of Compound (1) exhibits a characteristic X-ray powder diffraction (XRPD) pattern with characteristic peaks expressed in degrees 2θ (±0.2 degrees 2θ) at 5.4, 6.5, 8.7, 10.1, 11.9 13.0, 18.2, 20.2, and 24.7.

The characteristic peak positions and relative intensities for the XRPD pattern of the crystalline sodium salt form is shown in Table 2 below.

TABLE 2
Compound (1)
Crystalline Na Salt

| Angle | |
|---|---|
| 2-Theta ° | Rel. Intensity % |
| 5.4 | 42 |
| 6.5 | 29 |
| 8.7 | 43 |
| 10.1 | 100 |
| 11.9 | 39 |
| 13.0 | 52 |
| 18.2 | 51 |
| 20.2 | 42 |
| 24.7 | 30 |

The crystalline salt form, particularly sodium salt form, may be preferred for pharmaceutical formulation processing. In particular, the sodium salt form has certain properties making it particularly suitable for formulating in a Lipid-Based Drug Delivery System (LBDDS).

The sodium salt form was found to have much improved solubility in excipients commonly used for LBDDS formulation including, for example, propylene glycol and ethanol. The table below provides data demonstrating the much improved solubility of the sodium salt form of Compound (1) as compared to the Type A form of Compound (1) in particular excipients:

Comparison of Solubility of Compound (1) Na Salt Vs. Compound (1) Type A in Various Excipients

| | Compound (1) Na salt | Type A of Compound (1) |
|---|---|---|
| Excipient | (mg/mL) | (mg/mL) |
| PEG 400 | 233.6 ± 34 | 136.8 ± 3.2 |
| Propylene Glycol | >468 | 1.3 ± <0.01 |
| Ethanol | 187.0 ± 23.9 | 0.9 ± 0.1 |
| Capmul PG8 | <169 | 172.6 ± 8.3 |
| Capmul MCM | 262.5 ± 2.6 | 220.6 ± 7.4 |
| Transcutol P | 430.6 ± 14.7 | 24.3 ± 0.3 |
| Labrasol | 174.6 ± 11.8 | 146.7 ± 5.1 |

The much improved solubility of the sodium salt form in propylene glycol and ethanol makes this form particularly suited as the Compound (1) component in the compositions of the invention when such are used in the development of an LBDDS formulation employing one or more of these common excipients.

Second, the sodium salt unexpectedly exhibits higher form stability in propylene glycol and ethanol as compared to the Type A form. In particular, the Type A form of Compound (1) exhibits a clear form change when it is slurried in either ethanol or propylene glycol, as is demonstrated by a change in its XRPD pattern. By contrast, when the crystalline sodium salt form of Compound (1) is slurried in either propylene glycol or ethanol, there is no change in the XRPD pattern observed for the remaining solid phase. This demonstrates the improved stability of the sodium salt form in these excipients which, again, makes the sodium salt form particularly suited as the Compound (1) component in the compositions of the invention when such are used in the development of an LBDDS formulation employing one or more of these common excipients. The methods used in generating these results are described below in the Methods of Characterization section.

The above results obtained with the crystalline sodium salt are unexpected because it is generally not possible to predict such differences in solubility and any trend in physical stability between the free form and different salt forms of a compound, and in particular for Compound (1), even after such forms have been successfully prepared.

In an even more specific embodiment, the present invention is directed to the above-described methods and compositions wherein Compound (1) is in a crystalline sodium salt form that has at least the following characteristic: an X-ray powder diffraction pattern comprising a peak at 10.1 degrees 2θ (±0.2 degrees 2θ) when measured using CuKα radiation.

Another embodiment is directed to the above-described methods and compositions wherein Compound (1) is in a crystalline sodium salt form having an XRPD pattern comprising a peak at 10.1 degrees 2θ (±0.2 degrees 2θ) as described above and further comprising peaks at 13.0 and 18.2 degrees 2θ (±0.2 degrees 2θ) when measured using CuKα radiation.

Another embodiment is directed to the above-described methods and compositions wherein Compound (1) is in a crystalline sodium salt form having an XRPD pattern comprising a peak at 10.1 degrees 2θ (±0.2 degrees 2θ) as described above and further comprising peaks at 5.4, 8.7, 13.0 and 18.2 degrees 2θ (±0.2 degrees 2θ) when measured using CuKα radiation.

Another embodiment is directed to the above-described methods and compositions wherein Compound (1) is in a crystalline sodium salt form having an XRPD pattern comprising a peak at 10.1 degrees 2θ (±0.2 degrees 2θ) as described above and further comprising peaks at 5.4, 6.5, 8.7, 11.9, 13.0, 18.2, 20.2 and 24.7 degrees 2θ (±0.2 degrees 2θ) when measured using CuKα radiation.

Another embodiment is directed to the above-described methods and compositions wherein at least 50%, preferably at least 75%, more preferably at least 95%, more preferably at least 99%, of the Compound (1) component is present in the form of the crystalline salt, preferably sodium salt, of Compound (1) as may be characterized by any of the abovementioned XRPD-defined embodiments. The presence of such amounts of the crystalline salt of Compound (1) in a quantity of Compound (1) is typically measurable using XRPD analysis of the compound.

The crystalline salts of Compound (1) can be prepared by processes which comprise crystallizing Compound (1) from a solution in solvents under conditions which yield the crystalline salt. The precise conditions under which the crystalline salt is formed may be empirically determined and the following merely exemplify methods which have been found to be suitable in practice.

The crystalline sodium salt of Compound (1) may be prepared by a process comprising the following steps:
(i) dissolving compound (1) in an ketones or acetate solvents, optionally containing water as a co-solvent, by heating the mixture as a slurry or by obtaining a complete solution
(ii) adding water to the solution obtained in step (i) while maintaining the solution at a temperature of about 50-70° C. to obtain a solution or slurry;
(iii) seeding with the crystalline sodium salt of Compound (1)
(iv) cooling the slurry obtained in step (iii) to obtain solid material; (iv) collecting the solid material of step (iii) and drying said material at a temperature of about 45 to 75° C. to obtain the crystalline sodium salt of Compound (1). Other pharmaceutically acceptable salts may be prepared analogously.

Additional alternative processes for preparing the crystalline salts of Compound (1) may be found in the Examples section below.

Pharmaceutical Compositions and Methods

The above-described combination therapies using a Compound (1) or pharmaceutically acceptable salts thereof, and at least one of the following further HCV inhibiting compounds (A)-(U), are useful as for treating HCV infections in view of the demonstrated inhibitory activity of Compound (1) against HCV NS3 serine protease and the demonstrated HCV inhibitory activity of compounds (A)-(U) (see the above citations as to each compound). The combination therapy is therefore useful in treatment of HCV infection in a mammal and can be used for the preparation pharmaceutical compositions and kits for treating an HCV infection or alleviating one or more symptoms thereof in a patient. Although this combination therapy is expected to be effective against other HCV genotypes, including HCV genotypes 4, 5 and 6, treating HCV genotype 1 infection is preferred, including subgenotypes 1a and 1b. The individual active agents Compound (1) and compound (A) to (U) can be administered separately via separate pharmaceutical dosage forms, in either order or at the concurrently, or together as part of one pharmaceutical dosage form.

The appropriate dosage amounts and regimens for a particular patient can be determined by methods analgous to those known in the art and by reference to the disclosures in U.S. Pat. Nos. 6,323,180 and 7,585,845 for Compound (1) and to each of the patent and literature references referred to above in describing compounds (A)-(U), for example. Furthermore, to the extent that an interferon (e.g. a pegylated alpha interferon) and/or ribivarin might be included in the combination therapy of, reference can be made to the well known and approved dosage levels for these products.

Generally, a therapeutically effective amount for the treatment of HCV infection in the mammal is administered. In one embodiment, about 50 mg to about 1000 mg, more preferably from about 120 mg to about 480 mg, of the Compound (1) component is administered per adult human per day in single or multiple doses and an effective dose of the compound (A)-(U) component, as may be determined by reference to the above-cited literature, is administered per adult human per day in single or multiple doses. As described above, the dose or doses of the Compound (1) component and the compound (A)-(U) component can be administered together as a single composition or separately.

In another regimen according to the invention, a loading dose amount of Compound (1), or pharmaceutically acceptable salt thereof, and/or a loading dose amount of compound (A) to (U), or pharmaceutically acceptable salt thereof, is administered for the first administration dose of the treatment. The loading dose amount is higher than the dose amount administered for subsequent administrations in the treatment. Preferably, the loading dose amount is about double in quantity, by weight, of the amount in subsequent administrations in the treatment. For example, in one embodiment, the first dose of Compound (1) is administered at dosage of about 240 mg and subsequent doses of Compound (1) are administered at a dosage of about 120 mg, once or twice per day. In another embodiment, the first dose of Compound (1) is administered at a dosage of about 480 mg and subsequent doses of Compound (1) are administered at a dosage of about 240 mg, once or twice per day.

In additional embodiments of the loading dose regimen, Compound (1) or a pharmaceutically acceptable salt thereof is administered in a loading dose of 480 mg on day 1 and 240 mg/day on subsequent days, preferably by once daily administration (QD dosing). In an alternative of this loading dose regimen, the loading dose of Compound (1) or a pharmaceutically acceptable salt thereof is 480 mg in the first dose with subsequent doses of Compound (1) or a pharmaceutically acceptable salt thereof at 240 mg twice per day (BID dosing). In another alternative of the this loading dose regimen, the loading dose of Compound (1) or a pharmaceutically acceptable salt thereof on day 1 is 240 mg and the subsequent daily doses of Compound (1) or a pharmaceutically acceptable salt thereof are 120 mg/day preferably by once daily administration.

By using this loading dose concept, a clear advantage is that is it thereby possible to achieve steady state levels of active drug in the patient's system earlier than would otherwise be achieved. The blood level achieved by using a doubled loading dose is the same as would be achieved with a double dose but without the safety risk attendant to the subsequent continuous administration of a double dose. By reaching the targeted steady state level of active drug earlier in therapy also means that there less possibility of insufficient drug pressure at the beginning of therapy so that resistant viral strains have a smaller chance of emerging.

Specific optimal dosage and treatment regimens for any particular patient will of course depend upon a variety of factors, including the age, body weight, general health status, sex, diet, time of administration, rate of excretion, drug combination, the severity and course of the infection, the patient's disposition to the infection and the judgment of the treating physician. In general, the compound is most desirably administered at a concentration level that will generally afford antivirally effective results without causing any harmful or deleterious side effects.

Specific embodiments of the present invention include methods of treating HCV infection in a mammal comprising administering to the mammal therapeutically effective amounts of any of the below combinations:

(1) Compound (1)+compound (A)

(2) Compound (1)+compound (B)

(3) Compound (1)+compound (C)

(4) Compound (1)+compound (D)

(5) Compound (1)+compound (E), as PSI-7851 racemate or PSI 7977 optically pure form

(6) Compound (1)+compound (F)

(7) Compound (1)+compound (G)

(8) Compound (1)+compound (H)

(9) Compound (1)+compound (I)

(10) Compound (1)+compound (J)

(11) Compound (1)+compound (K)

(12) Compound (1)+compound (L)

(13) Compound (1)+compound (M)

(14) Compound (1)+compound (N)

(15) Compound (1)+compound (O)

(16) Compound (1)+compound (P)

(17) Compound (1)+compound (Q)

(18) Compound (1)+compound (R)

(19) Compound (1)+compound (S)

(20) Compound (1)+compound (T)

(21) Compound (1)+compound (U)

In each of the above embodiments, Compound (1) and/or the other anti-HCV compound (A) to (U) can be in the form of its pharmaceutically acceptable salt. As mentioned above, a preferred form of Compound (1) is as the sodium salt, which can be in crystalline form. Therefore, eighteen further individual embodiments of the invention include any of the above eighteen embodiments wherein Compound (1) is in the form of its sodium salt.

The doses of the Compound (1) component, the compound (A)-(U) component, and the optionally additional anti-HCV agent component, at a selected dosage level are typically administered to the patient via a single or separate pharmaceutical composition. See, e.g., the descriptions in U.S. Pat. Nos. 6,323,180 and 7,585,845 for examples of the various types of forms of compositions that may be employed in the present invention. The pharmaceutical composition(s) may be administered orally, parenterally or via an implanted reservoir. The term parenteral as used herein includes subcutaneous, intracutaneous, intravenous, intramuscular, intra-articular, intrasynovial, intrasternal, intrathecal, and intralesional injection or infusion techniques. Oral administration is a preferred administration, and in that embodiment all agents in the combination therapy are administered orally.

The pharmaceutical compositions of this invention may contain any conventional non-toxic pharmaceutically-acceptable carriers, diluents, adjuvants, excipients or vehicles. In some cases, the pH of the formulation may be adjusted with pharmaceutically acceptable acids, bases or buffers to enhance the stability of the formulated compound or its delivery form.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents.

The pharmaceutical compositions may also be in the form of separate oral pharmaceutical compositions of Compound (1), or pharmaceutically acceptable salt thereof, and one or more of the further HCV inhibiting compounds (A)-(U), or pharmaceutically acceptable salt thereof, and optionally additional anti-HCV agents, or a combined oral pharmaceutical composition of these components. The oral pharmaceutical compositions may be orally administered in any orally acceptable dosage form including, but not limited to, tablets, capsules (e.g., hard or soft gelatin capsules), including liquid-filled capsules, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. Examples of soft gelatin capsules that can be used include those disclosed in EP 649651 B1 and U.S. Pat. No. 5,985,321. When aqueous suspensions are administered orally, the active ingredient is combined with emulsifying and suspending agents. If desired, certain sweetening and/or flavoring and/or coloring agents may be added.

Other suitable vehicles or carriers for the above noted formulations and compositions can be found in standard pharmaceutical texts, e.g. in “Remington's Pharmaceutical Sciences”, 19^{th} ed., Mack Publishing Company, Easton, Pa., 1995.

With respect to Compound (1), one formulation type is a lipid-based pharmaceutical composition suitable for oral administration via a liquid- or semi-solid-filled capsule. This lipid-based pharmaceutical compositions constitutes a type of self-emulsifying drug delivery system (hereinafter “SEDDS”), and exhibits acceptable stability and bioavailability and is therefore particularly suited for the therapeutic delivery of Compound (1). The following is one general example of a liquid fill formulation of Compound (1) sodium salt for use in such a system:
(a) about 10% to 20% by weight of a Compound (1) as the sodium salt;
(b) about 40% to 50% by weight of a pharmaceutically acceptable lipid selected from monoglycerides of caprylic and capric fatty acids; diglycerides of caprylic and capric fatty acids, and mixtures thereof;
(c) about 25% to 35% by weight of a pharmaceutically acceptable hydrophilic surfactant selected from tocopheryl polyethylene glycol succinate, polyoxyl 40 hydrogenated castor oil, and polyoxyl 35 castor oil and mixtures thereof;
(d) about 5% to 10% by weight of a pharmaceutically acceptable hydrophilic solvent selected from propylene glycol, polyethylene glycol, ethanol, water, and mixtures thereof.

This fill composition may be prepared in a conventional manner, for example, by a method comprising mixing together the liquid components, e.g., the pharmaceutically acceptable lipid(s), surfactant(s) and solvent(s); optionally heating the mixture obtained if necessary to sufficiently melt one or more of the components of the mixture; adding the Compound (1) to the resulting mixture and further mixing until all or substantially all of the Compound (1) is solubilized, e.g. until the solution is visually clear. The resulting fill solution is then formulated into the desired dosage form, for example, capsules including hard shell or softgel capsules (e.g., hard or soft gelatin capsules), by known manufacturing technology. Examples of SEDDS capsule formulations are provided in the Examples section herein.

When compositions containing the a crystalline salt form of Compound (1) are formulated in a liquid vehicle, for example, as a liquid solution or suspension for oral administration or by injection, including for example in liquid-filled capsules, the salt will lose its crystalline nature. Nevertheless, the final liquid-based pharmaceutical composition will contain the salt of Compound (1) and therefore compositions containing such a salt are considered a separate embodiment embraced by the present invention. As discussed above, by using a method for preparing the salt, particularly sodium salt, in a stable crystalline form efficient pharmaceutical processing and pharmaceutical formulation manufacture using the salt form is facilitated.

Another embodiment is directed to a package comprising one or more pharmaceutically acceptable dosage forms containing a Compound (1) or a pharmaceutically acceptable salt thereof, and instructions directing the administration of Compound (1) or a pharmaceutically acceptable salt thereof and one or more of the further HCV inhibiting compounds (A)-(U), or a pharmaceutically acceptable salt thereof, for the treatment of HCV infection. The doses of Compound (1) are typically included as individual pharmaceutical dosage forms, e.g. tablets or capsules, and the package is typically a box containing these dosage forms (which dosage forms themselves may be contained in a bottle or blister pack, which is contained in the package). The instructions are typically included in a package insert document contained in the package, but may also be written on the outer package itself and/or on inner packaging. Further individual package embodiments of the invention include wherein the above-described package contains instructions directing the administration of Compound (1) or a pharmaceutically acceptable salt thereof and only one of the further HCV inhibiting compounds selected from compounds (A)-(U), or a pharmaceutically acceptable salt thereof, for the treatment of HCV infection.

Methods of Characterization

1. X-Ray Powder Diffraction

X-ray powder diffraction analyses were conducted on a Bruker AXS X-Ray Powder Diffractometer Model D8 Discover, available from Bruker AXS, Inc. of Madison, Wis., using CuKα radiation. The instrument is equipped with a long fine focus x-ray tube. The tube power was set to 40 kV and 40 mA. The instrument was operated in parallel beam mode with a Gobel Mirror, using a 0.6 mm exit slit, a 0.4° soller slit, a LiF flat crystal diffracted beam monochromator and a NaI scintillation detector. A detector scan was run using a tube angle of 1° 2θ. Step scans were run from 2 to 40° 2θ, at 0.05° per step, 4 sec per step. A reference quartz standard was used to check instrument alignment. Samples were prepared for analysis by filing a zero background quartz holder.

2. Solubility and Form Change Studies

The solubility of Compound (1), as either Type A or the sodium salt form, was investigated in various non-aqueous solvents. The solutions were prepared by addition of excess Compound (1) to 0.25 ml to 1.0 ml of excipient in amber screw cap vials with Teflon lined caps. The samples were allowed to rotate at room temperature for up to 4 days. Sampling was done by centrifuging (14,000 rpm on the Eppendorf model 5415C table top centrifuge) and filtering through a 0.45 μm PVDF filter. The filtrate was subject to HPLC analysis for determining the solubility. HPLC analysis was conducted with an Agilent 1100 using gradient or isocratic conditions. Both methods used acetonitrile/water (each with 0.1% Trifluoroacetic Acid) and an ACE C-18 stationary phase with column heating maintained at 40-45° C. The wavelength of detection was set at 220 nm or 264 nm. Wet solids were collected and analyzed for form change (stability) by XRPD.

XRPD analyses for the form change studies were conducted on a Bruker AXS X-Ray Powder Diffractometer Model D8 Discover or D8 Advance, available from Bruker AXS, Inc. of Madison, Wis., using CuKα radiation. The tube power was set to either 40 kV and 40 mA or 40 kV and 30 mA. The instrument(s) were operated in parallel beam mode with a Gobel Mirror, using a 0.6 mm exit slit with a 0.4° soller slit and LiF flat crystal diffracted beam monochromator or using 1 mm divergence slit with 0.12 mm soller slits. Bragg-Brentano configuration with the D8 Advance was also used for some analyses with 1 mm divergence slit with 0.12 mm soller slits. Each configuration/instrument employed NaI scintillation detector. Detector scans were run using a tube angle of 1° 2θ. Step scans were run from 2 to 35° or 40° 2θ, at 0.05° per step, with 0.6 or 4 seconds per step. A reference quartz standard was used to check instrument alignment. Samples were prepared for analysis by filing a zero background quartz holder or Ni plated holder.

In order that this invention be more fully understood, the following examples are set forth. These examples are for the purpose of illustrating embodiments of this invention, and are not to be construed as limiting the scope of the invention in any way. The reactants used in the examples below may be obtained either as described herein, or if not described herein, are themselves either commercially available or may be prepared from commercially available materials by methods known in the art. Certain starting materials, for example, may be obtained by methods described in the International Patent Applications WO 00/09543, WO 00/09558, WO 00/59929, U.S. Pat. Nos. 6,323,180, 6,608,027, 7,514,557 and 7,585,845.

Unless otherwise specified, solvents, temperatures, pressures, and other reaction conditions may be readily selected by one of ordinary skill in the art. Typically, reaction progress may be monitored by High Pressure Liquid Chromatography (HPLC), if desired, and intermediates and products may be purified by chromatography on silica gel and/or by recrystallization.

### EXAMPLES

### Methods for Preparing Compound (1) and Compound (1) Na Salt

Methods for preparing amorphous Compound (1) can be found in U.S. Pat. Nos. 6,323,180, 7,514,557 and 7,585,845, which are herein incorporated by reference. The following Examples 1 to 5 provide methods for preparing additional forms of Compound (1) that may be used in the present invention.

### Example 1

### Preparation of Type A Polymorph of Compound (1)

Amorphous Compound (1) (Batch 7, 13.80 g) was added to a 1000 ml three neck flask. Absolute ethanol (248.9 g) was added to the flask. While stirring, the contents of the flask were heated at 60 degrees C./hr to ˜74 degrees C. (Solids do not dissolve at 74 degrees C.). Water (257.4 g) was then added linearly over 4 hr to the resulting slurry while stirring and maintaining the temperature at 74 degrees C. After the water addition was complete, the temperature was reduced linearly to ambient temperature at 8 degrees C./hr and then held at ambient temperature for 6 hrs while stirring. The resulting solids were collected by filtration and washed with 50 ml of 1/1 (w/w) EtOH/Water. The wet solids were dried on the funnel for 30 minutes by sucking N₂ through the cake. (XRPD analysis on this sample indicates that the pattern is similar to the EtOH solvate). The solids were then dried at 65-70 degrees C. under vacuum (P=25 in Hg) and a nitrogen bleed for 1.5 hr. The resulting solids (12.6 g, 95.5% corrected yield) were confirmed by XRPD as being Type A Compound (1).

### Example 2

### Preparation of the Sodium Salt of Compound (1)—Method 1

2.1 g of amorphous sodium salt of Compound (1) and 8.90 g of acetone was added to a vial and stirred at ambient temperature for 3 hr. The slurry was filtered off mother liquors and the resulting solids were dried for 20 minutes under nitrogen flow for 20 minutes. 1.51 g of crystalline sodium salt of Compound (1) as solids was collected.

### Example 3

### Preparation of the Sodium Salt of Compound (1)—Method 2

15.6 g of Type A of Compound (1), 175 ml of acetone and 3.6 ml of water was added to a 250 ml reactor and heated to 53 degrees C. to dissolve the solids. 900 ul of 10.0 N NaOH was added to reactor and the solution was seeded with Type A. The seeded solution was stirred at 53 degrees C. for 10 minutes. A second 900 ul portion of 10.0 N NaOH was added and the system was stirred at 53 degrees C. for 30 minutes over which a slurry developed. The slurry was cooled to 19 degrees C. at a cooling rate of 15 degrees C. per hour and held overnight at 19 degrees C. The final resulting slurry was filtered and the wet solids were washed with 15 ml of acetone. Dried solids for 1 hr at 52 degrees C. under vacuum with a nitrogen flow and then exposed the solids to lab air for one hour. Collected 12.1 g of Compound (1) crystalline sodium salt solids.

### Example 4

### Preparation of the Sodium Salt of Compound (1)—Method 3

25.4 Kg of amorphous Compound (1), 228 L of THF and 11.1 Kg of 10 wt % NaOH (aq) was added to a reactor. The components were mixed at 25 degrees C. to dissolve all solids. The resulting solution was filtered and the reactor and filter was washed with 23 L of THF. 180 L of solvent was removed using atmospheric distillation at 65 degrees C. 195 L of MIBK was added and 166 L of solvent was removed by vacuum distillation at ˜44 degrees C. 161 L of MIBK and 0.41 Kg of water was added back to the reactor and the contents were heated to 70 degrees C. 255 g of Compound (1) sodium salt seeds were added at 70 degrees C. and 1.42 L of water was added over 1.5 hours. After the water addition the slurry was held at 70 degrees C. for 45 minutes and then cooled to 45 degrees C over 1 hr. The resulting slurried was filtered and washed with 64 L of MIBK containing ˜0.8 weight % water. The wet cake was dried at 55 degrees C. to give ˜25 Kg of crystalline sodium salt of Compound (1).

### Example 5

### Preparation of the Sodium Salt of Compound (1)—Method 4

2.00 g of amorphous Compound (1), 9.96 g of THF and 0.11 g of water was added to a reactor and stirred at ambient temperature to dissolve solids. 0.820 ml of 21 weight % NaOEt in ethanol was added drop-wise while stirring the solution to get solution A. 15.9 g of n-BuAc and 160 ul of water was added to a second reactor and heated to 65 degrees C. (solution B). 2.56 g of Solution A was added to Solution B at 65 degrees C. and the resulting mixture was seeded with 40 mg of Compound (1) sodium salt seeds. The seeded mixture was aged at 65 degrees C. for 45 minutes. 2.56 g of Solution B was added to Solution A and aged for 45 minutes in four separate intervals. After the final addition and aging, the slurry was cooled to 50 degrees C. over 1 hour and filtered. The wet cake was washed with 6 ml of n-BuAc containing 0.5 weight % water. The final solids were dried at 50 degrees C. under vacuum using a nitrogen purge. Compound (1) crystalline sodium salt solids were collected.

### Example 6

### Preparation of the Sodium Salt of Compound (1)—Method 5

At room temperature a solution of sodium ethoxide in ethanol (21 weight %; 306 ml) was added to a solution of Compound (1) (745 g) in THF (2000 ml) and water (76.5 ml) while stirring. After stirring for 30 minutes, the mixture was filtered and the filter was washed with THF (85 ml). The resulting solution was warmed to 65° C. and treated with filtered butyl acetate (6640 ml, optionally pre-warmed to 65° C.) within 30 minutes. Seeding crystals (0.50 g) were added, and the mixture was stirred at 65° C. for 2 hours, while crystallization starts after about 30 minutes. The suspension was cooled to 50° C. within 1 hour and stirred at this temperature for an additional hour. The title compound was isolated by filtration, washed with filtered butyl acetate (765 ml, optionally pre-warmed to 50° C.) and dried at 65° C. for about 16 h giving Compound (1) crystalline sodium salt (˜725 g).

Compound (1) Na Salt Capsule Formulations

Three different liquid fill formulations were manufactured, two of which were encapsulated in softgel capsules (SGC) and one encapsulated in a hard-shell capsule (HSC).

### Example 7

### Softgel Capsule Formulation #1

The composition of the liquid fill formulation:

| Ingredient | Monograph | Functionality | % w/w |
|---|---|---|---|
| Compound (1) Na salt | | API | 15.0 |
| Mono-, Diglycerides of | | Lipid | 46.3 |
| Caprylic/Capric Acid | | | |
| (Capmul ® MCM) | | | |
| Polyoxyl 35 Castor Oil | NF | Surfactant | 30.8 |
| (Cremophor ® EL) | | | |
| Propylene Glycol | USP | Solvent | 7.7 |
| DL-α-tocopherol | USP | Anti-oxidant | 0.2 |
| Total | | | 100.0 |

Two specific soft-gel capsule drug product formulations were prepared according to the above general Formulation #1, a 40 mg product and a 120 mg product:

| | | 40 mg | 120 mg |
|---|---|---|---|
| Ingredient | Function | mg/capsule | mg/capsule |
| Compound (1) Na salt | Drug | 42.30¹ | 126.90² |
| (milled) | substance | | |
| Mono/Diglycerides of | Lipid phase | 130.57 | 391.70 |
| Caprylic/Capric Acid | | | |
| Polyoxyl 35 Castor Oil | Surfactant | 86.86 | 260.57 |
| (NF) | | | |
| Macrogolglycerol | | | |
| Ricinoleate (Ph. Eur.) | | | |
| Propylene Glycol | Solvent | 21.71 | 65.14 |
| Vitamin E (dl-alpha | Anti- | 0.56 | 1.69 |
| tocopherol) (USP) | oxidant | | |
| All-rac-alpha-tocopherol | | | |
| (Ph. Eur.) | | | |
| Nitrogen³ | Processing | q.s. | q.s. |
| | aid | | |
| Total Fill Weight | | 282.00 | 846.00 |
| Soft Gelatin Capsule | Shell | 280⁴ | 590⁵ |
| Shell | | | |
| Wet Total Capsule | | 562 | 1436 |
| Weight | | | |
| Dry Total Capsule | | 480 | 1250 |
| Weight | | | |

¹42.30 mg of Compound (1) Na salt is equivalent to 40.0 mg of the active moiety.
²126.90 mg of Compound (1) Na salt is equivalent to 120.0 mg of the active moiety.
³Nitrogen is used as a processing aid and does not appear in the final product.
⁴The approximate weight of the capsule shell before drying and finishing is 280 mg. The approximate weight of the capsule shell after drying and finishing is 198 mg.
⁵The approximate weight of the capsule shell before drying and finishing is 590 mg. The approximate weight of the capsule shell after drying and finishing is 404 mg.

### Example 8

### Softgel Caspule Formulation #2

The composition of the liquid fill formulation:

| Ingredient | Monograph | Functionality | % w/w |
|---|---|---|---|
| Compound (1) Na salt | | API | 15.0 |
| Mono-, Diglycerides of | | Lipid | 42.4 |
| Caprylic/Capric Acid | | | |
| (Capmul ® MCM) | | | |
| Polyoxyl 35 Castor Oil | NF | Surfactant | 33.9 |
| (Cremophor ® EL) | | | |
| Propylene Glycol | USP | Solvent | — |
| Oleic Acid | | Lipid | 8.5 |
| DL-α-tocopherol | USP | Anti-oxidant | 0.2 |
| Total | | | 100.0 |

A specific 150 mg soft-gel capsule drug product formulation was prepared according to the above general formula.

### Example 9

### Hard Shell Caspule Formulation #3

The composition of the liquid fill formulation:

| Ingredient | Monograph | Functionality | % w/w |
|---|---|---|---|
| Compound (1) Na salt | | API | 20.0 |
| Mono-, Diglycerides of | | Lipid | 53.8 |
| Caprylic/Capric Acid | | | |
| (Capmul ® MCM) | | | |
| Polyoxyl 35 Castor Oil | NF | Surfactant | 23.0 |
| (Cremophor ® EL) | | | |
| Propylene Glycol | USP | Solvent | 3.0 |
| DL-α-tocopherol | USP | Anti-oxidant | 0.2 |
| Total | | | 100.0 |

A specific 150 mg hard-shell capsule drug product formulation was prepared according to the above general formula.

Preparation of Formulations 1-3:

The drug substance is jet-milled to remove large aggregates so that the mixing time for the bulk fill manufacturing will be consistent and reasonably short. The target particle size distribution of the drug substance is to reduce the ×90 (v/v) to no more than 10 micron and the ×98 (v/v) to no more than 20 micron as measured by Sympatec. All the excipients in the fill formulation are combined in a mixing vessel and mixed until uniform prior to adding the drug substance. After addition of the drug substance, mixing continues until the fill solution is clear by visual inspection. A nitrogen blanket over the fill solution is used throughout the preparation as a standard practice. The fill solution is passed through a filter to remove any extraneous particles. Encapsulation of the filtered bulk fill material in capsules is performed utilizing standard soft gelatin or hard gelatin capsule technology and in-process controls. Filled capsules are dried and then washed with a finishing/wash solution prior to packaging resulting in shiny, pharmaceutically elegant capsules.

We claim:

1. A method of treating Hepatitis C viral infection in a mammal comprising administering to said mammal a therapeutically effective amount of:
a Compound (1): 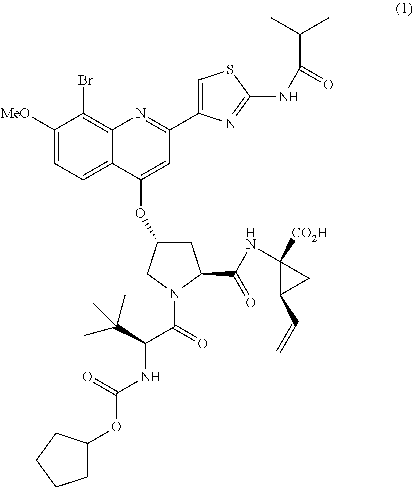 [SMILES: C=C[C@@H]1C[C@]1(NC(=O)[C@@H]1C[C@@H](Oc2cc(-c3csc(NC(=O)C(C)C)n3)nc3c(Br)c(OC)ccc23)CN1C(=O)[C@@H](NC(=O)OC1CCCC1)C(C)(C)C)C(=O)O]
or a pharmaceutically acceptable salt thereof,
and, either separately or together, at least one further HCV inhibiting compound selected from the group consisting of compounds (A) to (U), or a pharmaceutically acceptable salt thereof:

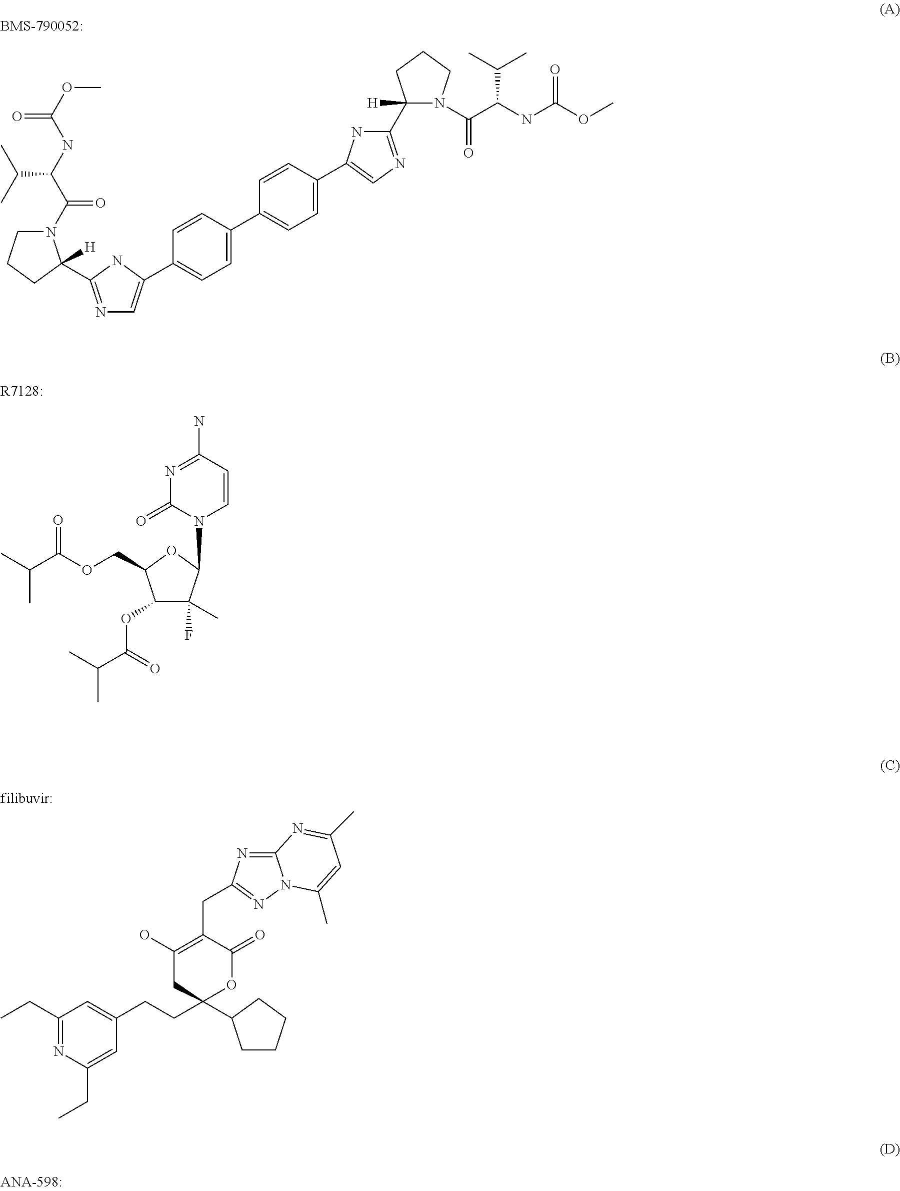

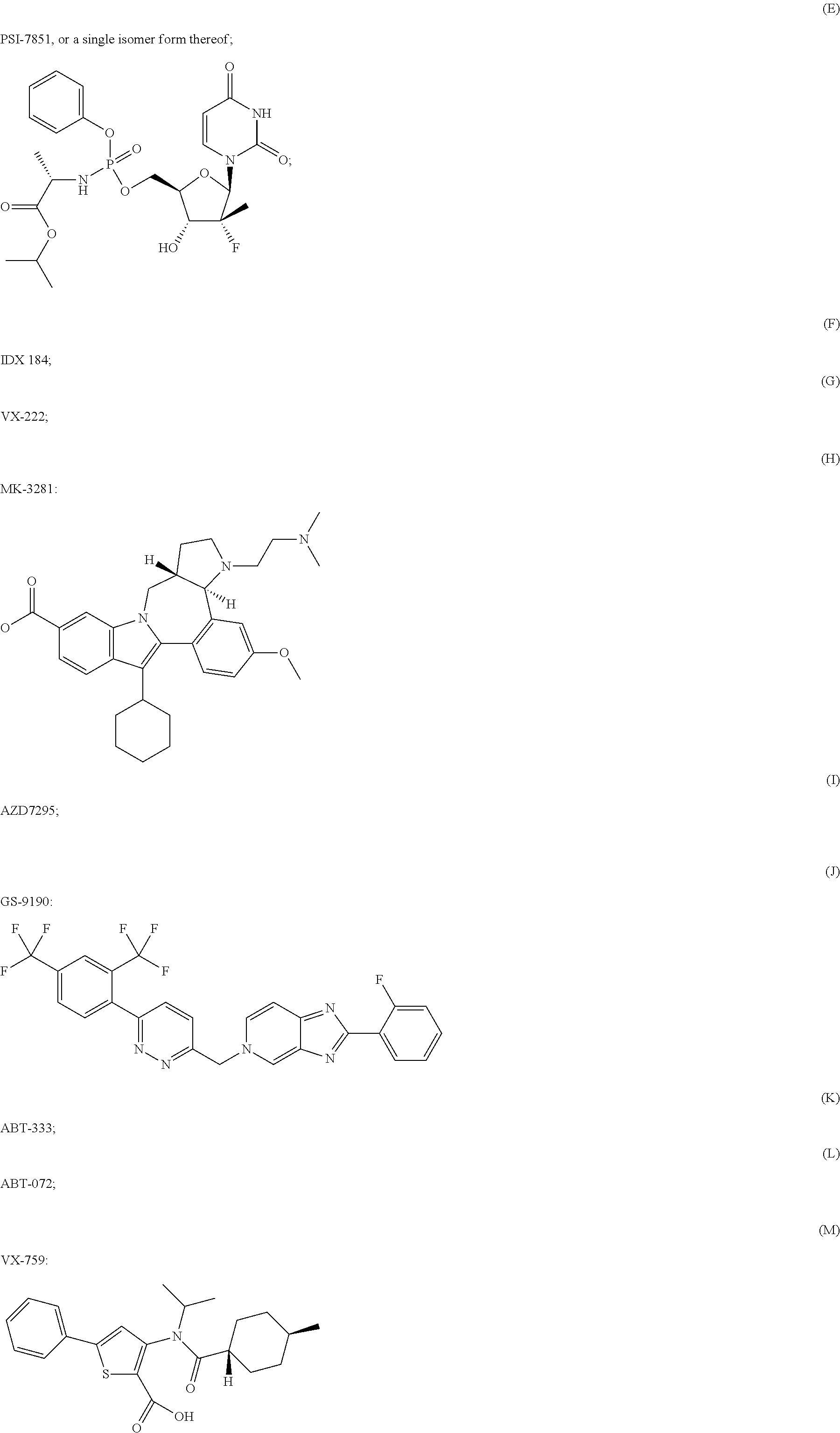

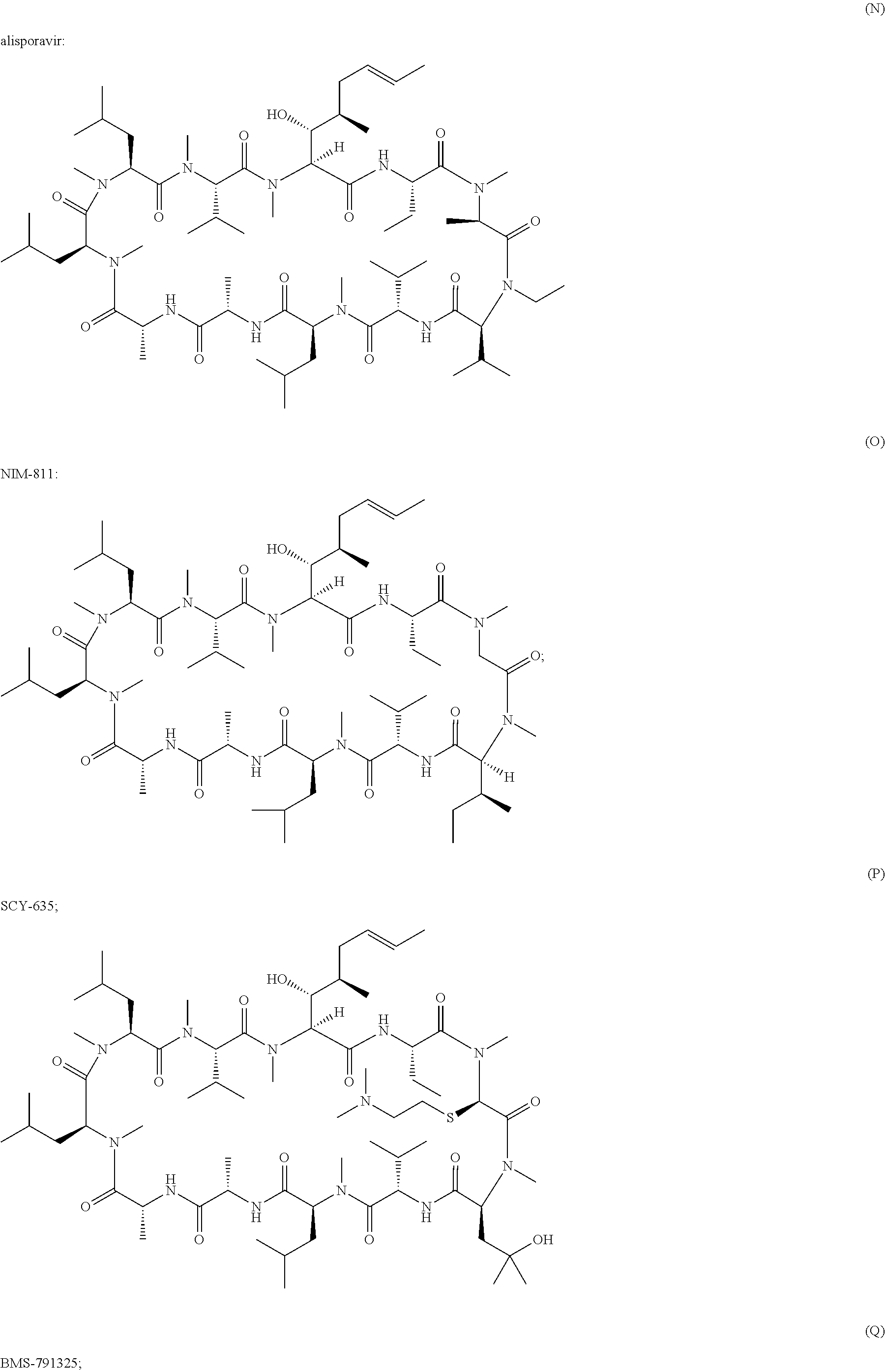

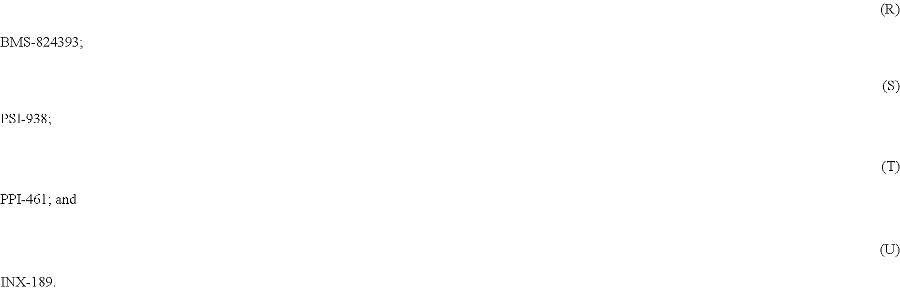

2. The method of claim 1 wherein the further HCV inhibiting compound is 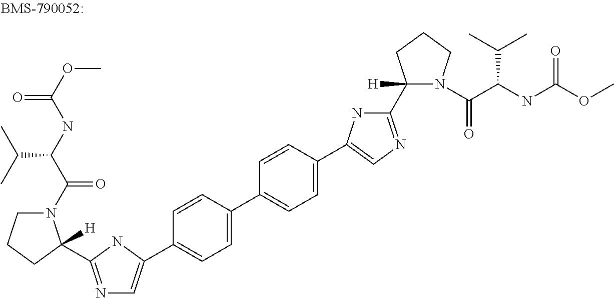 [SMILES: COC(=O)N[C@H](C(=O)N1CCC[C@H]1c1ncc(-c2ccc(-c3ccc(-c4cnc([C@@H]5CCCN5C(=O)[C@@H](NC(=O)OC)C(C)C)[nH]4)cc3)cc2)[nH]1)C(C)C]
or a pharmaceutically acceptable salt thereof.

3. The method of claim 1 wherein the further HCV inhibiting compound is R7128: 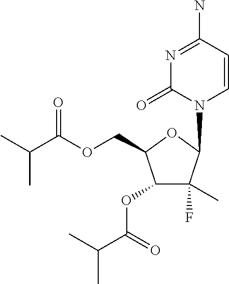 [SMILES: CC(C)C(=O)OC[C@H]1O[C@@H](n2ccc(N)nc2=O)[C@](C)(F)[C@@H]1OC(=O)C(C)C]
or a pharmaceutically acceptable salt thereof.

4. The method of claim 1 wherein the further HCV inhibiting compound is filibuvir: 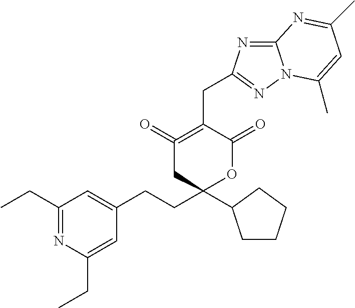
or a pharmaceutically acceptable salt thereof.

5. The method of claim 1 wherein the further HCV inhibiting compound is MK-3281: 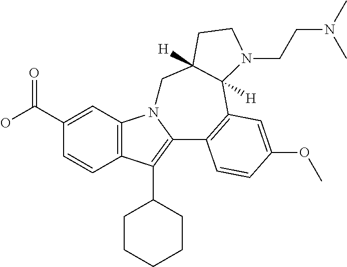 [SMILES: COc1ccc2c(c1)[C@H]1[C@@H](CCN1CCN(C)C)Cn1c-2c(C2CCCCC2)c2ccc(C(=O)O)cc21]
or a pharmaceutically acceptable salt thereof.

6. The method of claim 1 wherein the further HCV inhibiting compound is GS-9190: 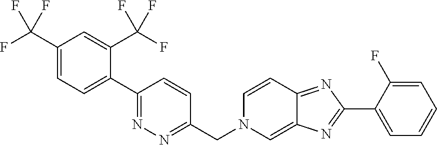 [SMILES: Fc1ccccc1-c1nc2ccn(Cc3ccc(-c4ccc(C(F)(F)F)cc4C(F)(F)F)nn3)cc-2n1]
or a pharmaceutically acceptable salt thereof.

7. The method of claim 1 wherein the further HCV inhibiting compound is VX-759: 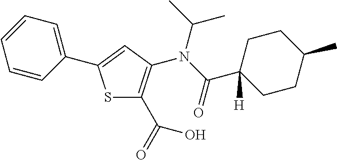 [SMILES: CC(C)N(c1cc(-c2ccccc2)sc1C(=O)O)C(=O)[C@H]1CC[C@H](C)CC1]
or a pharmaceutically acceptable salt thereof.

8. The method of claim 1 wherein the further HCV inhibiting compound is alisporavir: 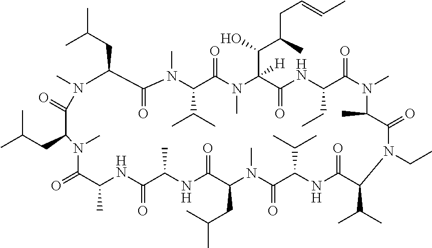 [SMILES: C/C=C/C[C@@H](C)[C@@H](O)[C@H]1C(=O)N[C@@H](CC)C(=O)N(C)[C@H](C)C(=O)N(CC)[C@@H](C(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
or a pharmaceutically acceptable salt thereof.

9. The method claim 1 wherein the further HCV inhibiting compound is NIM-811: 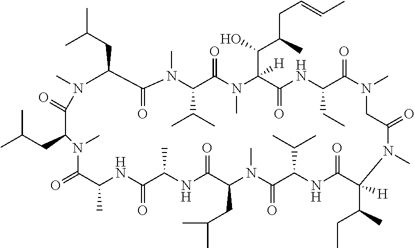 [SMILES: C/C=C/C[C@@H](C)[C@@H](O)[C@H]1C(=O)N[C@@H](CC)C(=O)N(C)CC(=O)N(C)[C@@H]([C@@H](C)CC)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
or a pharmaceutically acceptable salt thereof.

10. The method of claim 1 wherein the further HCV inhibiting compound is SCY-635: 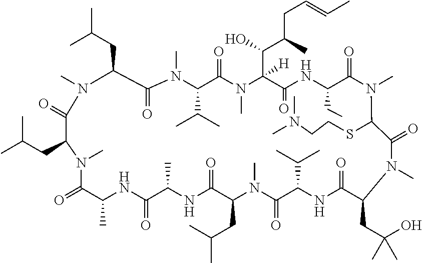 [SMILES: C/C=C/C[C@@H](C)[C@@H](O)[C@H]1C(=O)N[C@@H](CC)C(=O)N(C)C(SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
or a pharmaceutically acceptable salt thereof.

11. A package comprising one or more pharmaceutically acceptable dosage forms containing a Compound (1): 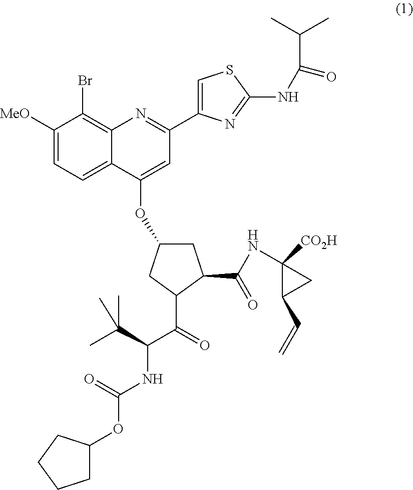 [SMILES: C=C[C@@H]1C[C@]1(NC(=O)[C@@H]1C[C@@H](Oc2cc(-c3csc(NC(=O)C(C)C)n3)nc3c(Br)c(OC)ccc23)CC1C(=O)[C@@H](NC(=O)OC1CCCC1)C(C)(C)C)C(=O)O]
or a pharmaceutically acceptable salt thereof, and instructions directing the administration of Compound (1) or a pharmaceutically acceptable salt thereof, and at least one further HCV inhibiting compound selected from the group consisting of compounds (A)-(U): 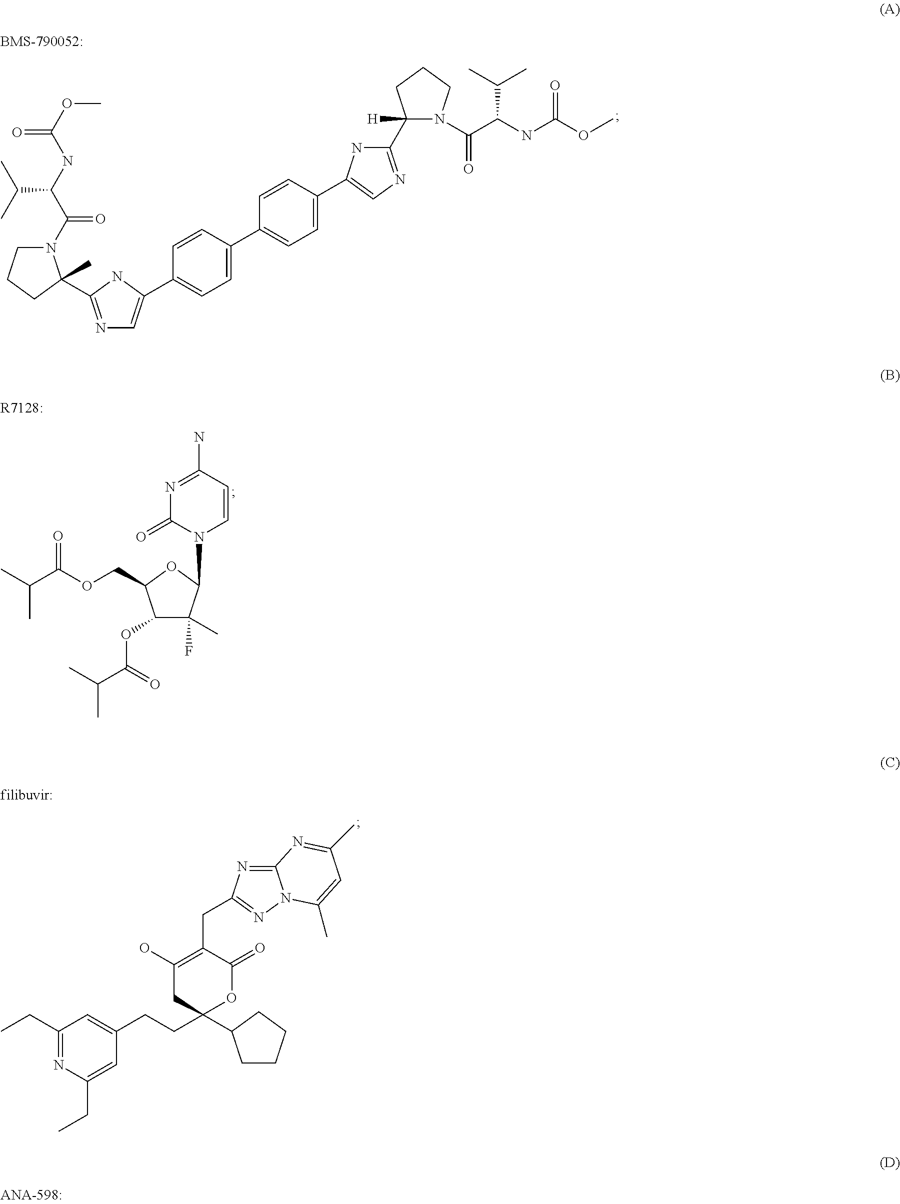 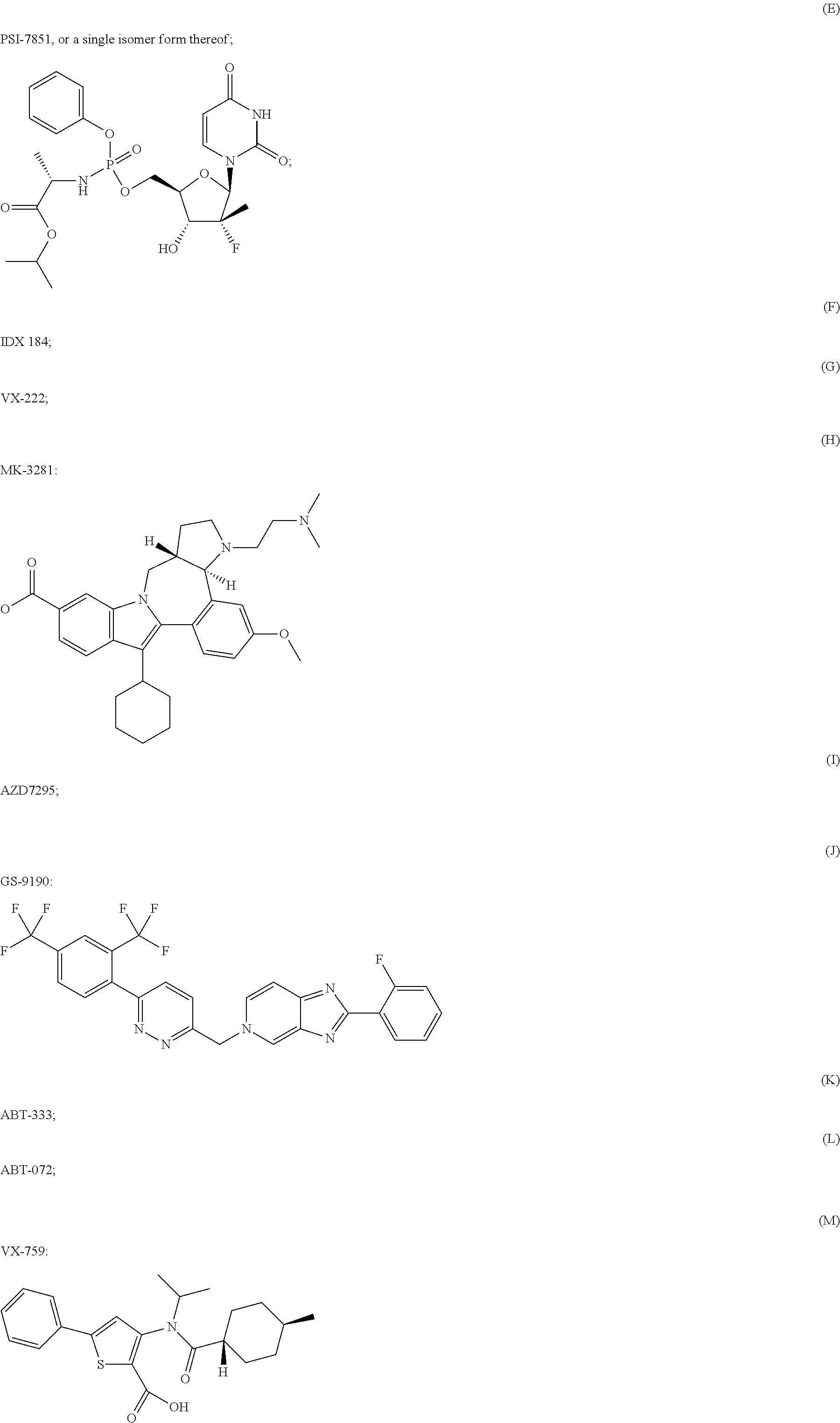 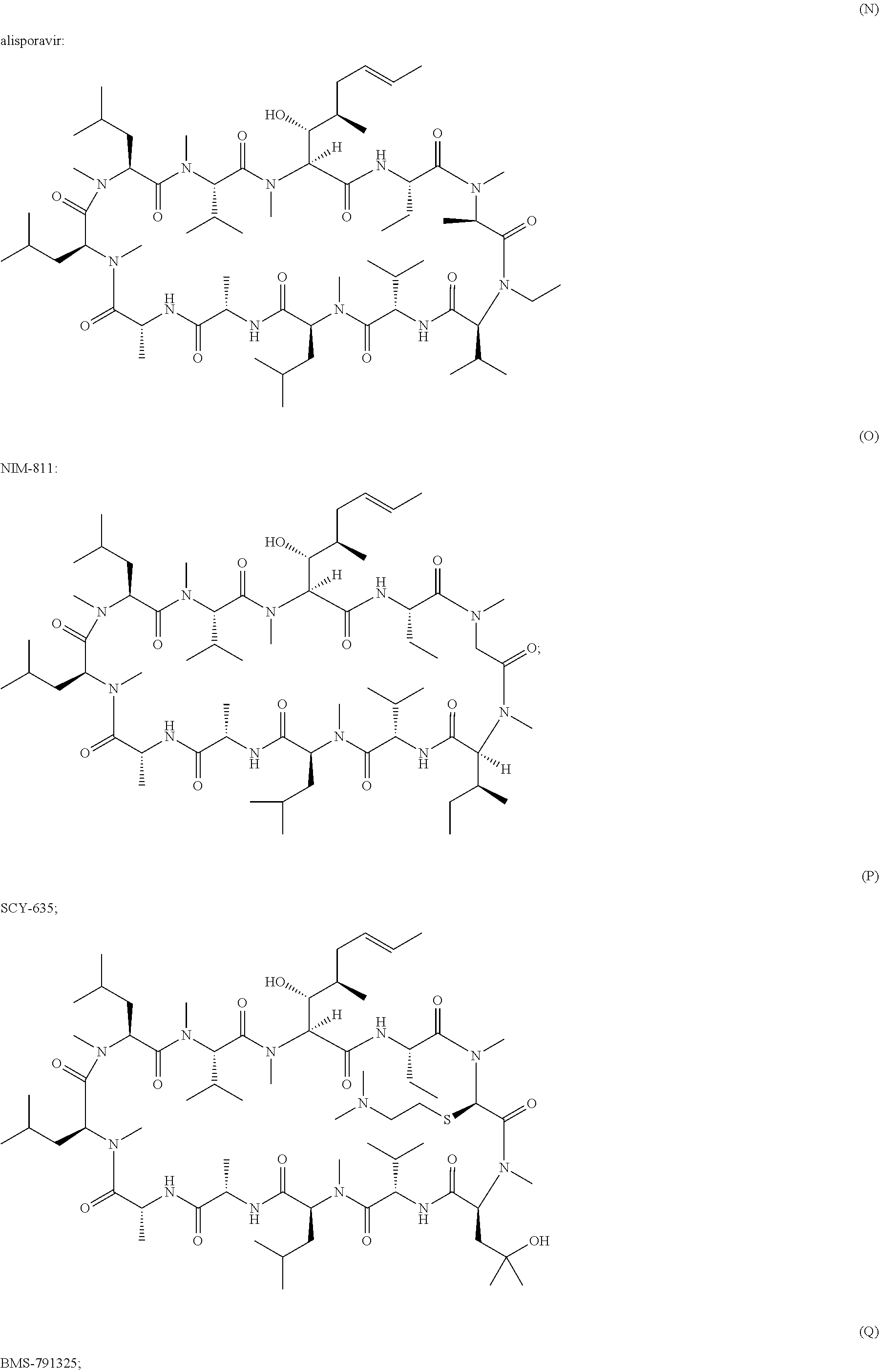 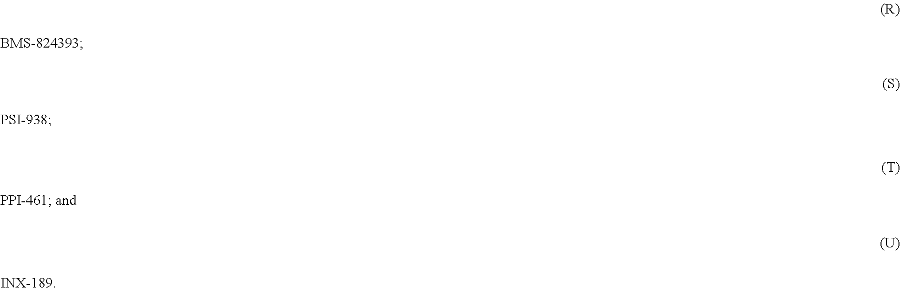
or pharmaceutically acceptable salt thereof, for the treatment of HCV infection.

12. The package of claim 11 wherein the further HCV inhibiting compound is BMS 790052: 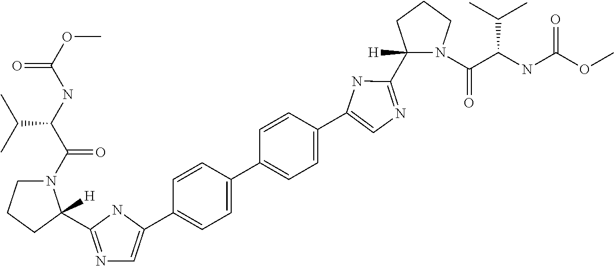 [SMILES: COC(=O)N[C@H](C(=O)N1CCC[C@H]1c1ncc(-c2ccc(-c3ccc(-c4cnc([C@@H]5CCCN5C(=O)[C@@H](NC(=O)OC)C(C)C)[nH]4)cc3)cc2)[nH]1)C(C)C]
or pharmaceutically acceptable salt thereof, for the treatment of HCV infection.

13. The package of claim 11 wherein the further HCV inhibiting compound is R7128: 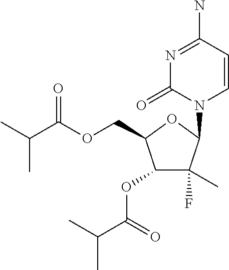 [SMILES: CC(C)C(=O)OC[C@H]1O[C@@H](n2ccc(N)nc2=O)[C@](C)(F)[C@@H]1OC(=O)C(C)C]
or pharmaceutically acceptable salt thereof, for the treatment of HCV infection.

14. The package of claim 11 wherein the further HCV inhibiting compound is filibuvir: 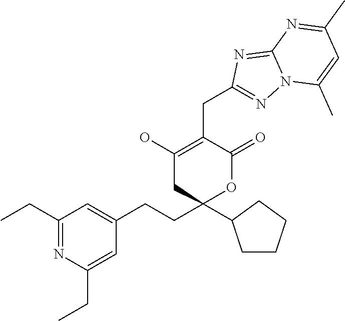 [SMILES: CCc1cc(CC[C@]2(C3CCCC3)CC(O)=C(Cc3nc4nc(C)cc(C)n4n3)C(=O)O2)cc(CC)n1]
or pharmaceutically acceptable salt thereof, for the treatment of HCV infection.

15. The package of claim 11 wherein the further HCV inhibiting compound is MK-3281: 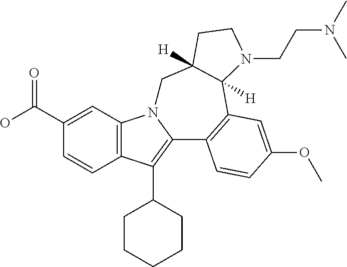 [SMILES: COc1ccc2c(c1)[C@H]1[C@@H](CCN1CCN(C)C)Cn1c-2c(C2CCCCC2)c2ccc(C(=O)O)cc21]
or pharmaceutically acceptable salt thereof, for the treatment of HCV infection.

16. The package of claim 11 wherein the further HCV inhibiting compound is GS-9190: 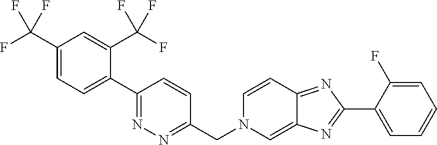 [SMILES: Fc1ccccc1-c1nc2ccn(Cc3ccc(-c4ccc(C(F)(F)F)cc4C(F)(F)F)nn3)cc-2n1]
or pharmaceutically acceptable salt thereof, for the treatment of HCV infection.

17. The package of claim 11 wherein the further HCV inhibiting compound is VX-759: 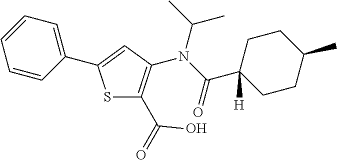 [SMILES: CC(C)N(c1cc(-c2ccccc2)sc1C(=O)O)C(=O)[C@H]1CC[C@H](C)CC1]
or pharmaceutically acceptable salt thereof, for the treatment of HCV infection.

18. The package of claim 11 wherein the further HCV inhibiting compound is alisporivir: 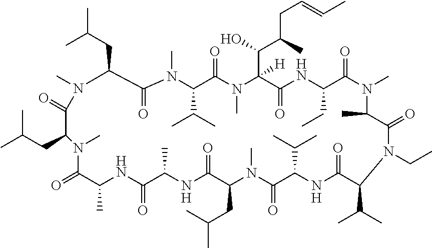 [SMILES: C/C=C/C[C@@H](C)[C@@H](O)[C@H]1C(=O)N[C@@H](CC)C(=O)N(C)[C@H](C)C(=O)N(CC)[C@@H](C(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
or pharmaceutically acceptable salt thereof, for the treatment of HCV infection.

19. The package of claim 11 wherein the further HCV inhibiting compound NIM-811: 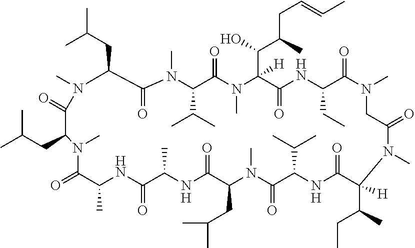 [SMILES: C/C=C/C[C@@H](C)[C@@H](O)[C@H]1C(=O)N[C@@H](CC)C(=O)N(C)CC(=O)N(C)[C@@H]([C@@H](C)CC)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
or pharmaceutically acceptable salt thereof, for the treatment of HCV infection.

20. The package of claim 11 wherein the further HCV inhibiting compound is SCY-635: 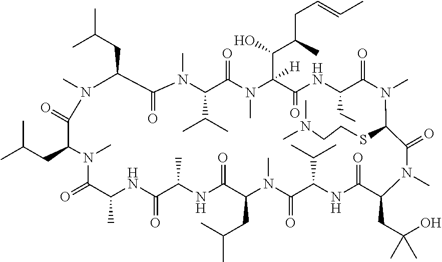 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
or pharmaceutically acceptable salt thereof, for the treatment of HCV infection.
